SEQUENCE LISTING (3 sequences)

SEQ ID NO: 1 - protein, 6 aa, Artificial
  Artificial peptide
DGAXLX

SEQ ID NO: 2 - protein, 6 aa, Artificial
  Artificial peptide
XZZZZX

SEQ ID NO: 3 - protein, 8 aa, Artificial
  Artificial peptide
DGAXLXXX

(19) United States
(12) Patent Application Publication
(10) Pub. No.: US 2010/0004180 A1
(43) Pub. Date: Jan. 7, 2010

(54) Agents for the Detection and Modulation of B-Cells and Antibodies

(76) Inventors: Jon A. Wolff, Shorewood, WI (US); Alex V. Sokoloff, Madison, WI (US)

Correspondence Address: FULBRIGHT & JAWORSKI L.L.P., 600 CONGRESS AVE., SUITE 2400, AUSTIN, TX, 78701, US

(21) Appl. No.: 12/432,285

(22) Filed: Apr. 29, 2009

Related U.S. Application Data
(60) Provisional application No. 61/048,755, filed on Apr. 29, 2008.

(51) Int. Cl. C07K 5/06; A61K 38/05; A61K 38/06; A61K 38/07; A61K 38/08; C07K 5/08; C07K 5/10; C07K 7/06; A01N 1/02; G01N 33/53

(52) U.S. Cl. 514/15; 514/19; 514/18; 514/17; 514/16; 530/331; 530/330; 530/329; 530/328; 435/2; 435/7.1

(57) ABSTRACT

The present invention relates to the identification and use of peptide and peptide analogs that represent epitopes for naturally-occurring antibodies (Nabs). These epitopes may be used to monitor the degree of autoimmunity in an organism, as well as to modulate the activity of B-cells producing Nabs.

The present application claims benefit of priority to U.S. Ser. No. 61/048,755, filed Apr. 29, 2008, the entire contents of which is hereby incorporated by reference.

### GOVERNMENT SUPPORT CLAUSE

This invention was made with government support under grant no. NS39915 awarded by the National Institutes of Health. The government has certain rights in the invention.

### BACKGROUND OF THE INVENTION

1. Field of the Invention

The present invention relates to fields of pathology, cellular biology and immunology. More particularly, the present invention relates to peptide mimitopes that are recognized by natural antibodies in serum. Methods for their use in the treatment of immune disorders also are provided.

2. Description of Related Art

Natural or naturally-occurring IgM Abs (Nabs) are important components of the innate immune system that participate in clearing pathogens, enhancing immune responses and preventing autoimmunity (Baumgarth et al., 2000; Boes, 2000; Ehrenstein et al., 1998; 2000; Ochsenbein et al., 1999). The molecular recognition mechanisms underlying the functions of natural IgM Abs are only understood to a limited degree (Notkins, 2004). This shortcoming is largely due to the fact that the majority of monoclonal natural IgM Abs with detectable binding activity in vitro are polyspecific and recognize a variety of Ags that share no obvious structural similarity (Dighiero et al., 1983; Notkins, 2004). Polyspecificity correlates with the encoding of Abs by germline genes and the structure of the CDR3_{H} region (Chen et al., 1991; Diaw et al., 1997). Kinetic and crystallographic data indicate that polyspecific Abs may accommodate unrelated Ags due to the high conformational plasticity of their antigen-combining sites (Foote and Milstein, 1994; James et al., 2003).

It is generally believed that polyspecificity is an inherent property of natural Abs, which enables a limited set of immunoglobulins to react with a large variety of pathogens degrade or modify endogenous Ags. An alternative view is that natural Abs with originally narrow specificity but high conformational plasticity may become polyspecific as a result of exposure to mild denaturing conditions. Such conditions include the presence of chaotropic agents, low pH, high salt, and oxidative physiological compounds (Bouvet et al., 2001; Dimitrov et al., 2006; McIntyre, 2004; McMahon and O'Kennedy, 2000). The concept of induced polyspecificity suggests that polyspecific Abs may be generated in the inflammatory environment induced by pathogen invasion or tissue destruction (Bouvet et al., 2001; Dimitrov et al., 2006; McIntyre, 2004).

The polyspecificity of natural Abs was historically studied using simple buffer media, such as buffered saline containing BSA or gelatin and non-ionic detergent (Dighiero et al., 1983). In view of the high conformational plasticity of natural Abs, it would appear that the absence of normal serum components, including those contributing to the maintenance of the physiological redox potential, might by itself lead to Abs conformational changes supporting polyspecificity (Foote and Milstein, 1994; James et al., 2003). It is also conceivable that low-affinity natural IgM Abs could more effectively react with surface Ags in buffer than in undiluted plasma or serum. Thus, additional studies are required to fully understand Ab polyspecificity and the requirements for Ag binding thereto.

### SUMMARY OF THE INVENTION

Thus, in accordance with the present invention, there is provided a method for inhibiting the binding of an antibody to an antigen in vivo comprising administering to a subject at least one peptide selected from the group consisting XL(X)ₘ, (X)ₘTP(X)ₙ (SEQ ID NO:1), (X)ₒY₁Y₂Y₃Y₄Y₅(X)ₚ (SEQ ID NO:2) and (x)ₒZ₁Z₂Z₃Z₄(X)ₚ (SEQ ID NO:3), or an analog thereof, wherein X is any amino acid; Y₁-Y₅ are each any amino acid, so long as at least one of Y₁-Y₅ is a W residue, and the remain of Y₁-Y₅ comprises (i) Y, F or H residue, and (ii) D or E residue; Z₁-Z₄ are each any amino acid, so long as at least one of Z₁-Z₄ is a W residue, and the remaining of Z₁-Z₄ comprises one D residue, two D residue's, one E residue, two E residue's, or one D and one E residue; m and n are independently 0-3, but n+m must be two or greater; and o and p are independently 0-2. The analog may be a peptidomimetic, and in particular, the analog is indole-3-propionic acid.

The peptide may have the formula (X)ₒZ₁Z₂Z₃Z₄(X)ₚ, and wherein the peptide comprises one D or E residue, and further comprises at one Y, F or H residue, and this peptide may in some embodiments further comprise another second Y, F or H residue selected independent of the first Y, F or H residue. The peptide may have the formula (X)ₒY₁Y₂Y₃Y₄Y₅(X)ₚ, and wherein the W residue is adjacent to the Y, F or H residue, and in some embodiments, the D or E residue is adjacent to the W residue or the Y, F or H residue. The method may further comprising administering to the subject at least two different peptides of the formula XLXXX (SEQ ID NO:4), or at least two different peptides of the formula (X)ₘTP(X)ₙ, or at least two different peptides of the formula (X)ₒZ₁Z₂Z₃Z₄(X)ₚ, or at least two different peptides of the formula (X)ₒY₁Y₂Y₃Y₄Y₅(X)ₚ, or a peptide of the formula of the XLXXX and a peptide of the formula (X)ₒZ₁Z₂Z₃Z₄(X)ₚ, or a peptide of the formula of the XLXXX and a peptide of the formula (X)ₒY₁Y₂Y₃Y₄Y₅(X)ₚ, or a peptide of the formula of the (X)ₒY₁Y₂Y₃Y₄Y₅(X)ₚ and a peptide of the formula (X)ₒZ₁Z₂Z₃Z₄(X)ₚ, or a peptide of the formula of the (X)ₒY₁Y₂Y₃Y₄Y₅(X)ₚ and a peptide of the formula (X)ₘTP(X)ₙ, or a peptide of the formula of the XLXXX and a peptide of the formula (X)ₘTP(X)ₙ, or a peptide of the formula of the (X)ₒZ₁Z₂Z₃Z₄(X)ₚ and a peptide of the formula (X)ₘTP(X)ₙ, or a peptide of the formula of the (X)ₒZ₁Z₂Z₃Z₄(X)ₚ, a peptide of the formula (X)ₘTP(X)ₙ, a peptide of the formula (X)ₒY₁Y₂Y₃Y₄Y₅(X)ₚ, and a peptide of the formula XLXXX.

The subject may suffer from an autoimmune disease, such as SLE or rheumatoid arthritis. The method may also further comprise administering to the subject an anti-inflammatory or immunosuppressive agent other than the peptide. The peptide may be delivered intravenously, intra-arterially, or subcutaneously. The administration is repeated, including 2, 3, 4, 5, 6, 7, 8, 9, 10 or more times. Administration may comprise delivery of an extended release formulation to the subject.

In another embodiment, there is provided an isolated and purified peptide selected from the group consisting XL(X)ₘ, (X)ₘTP(X)ₙ (SEQ ID NO:1), (X)ₒY₁Y₂Y₃Y₄Y₅(X)ₚ (SEQ ID NO:2) and (X)ₒZ₁Z₂Z₃Z₄(X)ₚ (SEQ ID NO:3), or an analog thereof, wherein X is any amino acid; Z₁-Z₄ are each any amino acid, so long as at least one of Z₁-Z₄ is W, and the remaining of Z₁-Z₄ include one D, two D's, one E, two E's, or one D and one E; m and n are independently 0-3, but n+m must be two or greater; and o and p are independently 0-2.

In yet another embodiment, there is provided a composition of matter comprising a peptide selected from the group consisting XL(X)ₘ, (X)ₘTP(X)ₙ (SEQ ID NO:1), (X)ₒY₁Y₂Y₃Y₄Y₅(X)ₚ (SEQ ID NO:2) and (X)ₒZ₁Z₂Z₃Z₄(X)ₚ (SEQ ID NO:3), or an analog thereof, wherein X is any amino acid; Z₁-Z₄ are each any amino acid, so long as at least one of Z₁-Z₄ is W, and the remaining of Z₁-Z₄ include one D, two D's, one E, two E's, or one D and one E; m and n are independently 0-3, but n+m must be two or greater; and o and p are independently 0-2, the peptide bound to a support. The support may be a bead, a membrane, a wafer, a bag, a filament, a dipstick, a microtiter plate or well thereof, or a chip.

In still another embodiment, there is provided an article of manufacture comprising (a) blood or plasma; (b) a container for holding the blood or plasma; and (c) at least one peptide selected from the group consisting XL(X)ₘ, (X)ₘTP(X)ₙ (SEQ ID NO:1), (X)ₒY₁Y₂Y₃Y₄Y₅(X)ₚ (SEQ ID NO:2) and (X)ₒZ₁Z₂Z₃Z₄(X)ₚ (SEQ ID NO:3), or an analog thereof bound to the inner surface of the container, wherein X is any amino acid; Z₁-Z₄ are each any amino acid, so long as at least one of Z₁-Z₄ is W, and the remaining of Z₁-Z₄ include one D, two D's, one E, two E's, or one D and one E; m and n are independently 0-3, but n+m must be two or greater; and o and p are independently 0-2.

In a further embodiment, there is provided a method for suppressing the secretion of a natural antibody in a subject comprising administering to the subject at least one peptide selected from the group consisting XL(X)ₘ, (X)ₘTP(X)ₙ (SEQ ID NO:1), (X)ₒY₁Y₂Y₃Y₄Y₅(X)ₚ (SEQ ID NO:2) and (X)ₒZ₁Z₂Z₃Z₄(X)ₚ (SEQ ID NO:3), or an analog thereof, wherein X is any amino acid; Z₁-Z₄ are each any amino acid, so long as at least one of Z₁-Z₄ is W, and the remaining of Z₁-Z₄ include one D, two D's, one E, two E's, or one D and one E; m and n are independently 0-3, but n+m must be two or greater; and o and p are independently 0-2.

In still a further embodiment, there is provided a method of removing a natural antibody from an antibody-containing fluid sample comprising contacting the sample with at least one peptide selected from the group consisting XL(X)ₘ, (X)ₘTP(X)ₙ (SEQ ID NO:1), (X)ₒY₁Y₂Y₃Y₄Y₅(X)ₚ (SEQ ID NO:2) and (X)ₒZ₁Z₂Z₃Z₄(X)ₚ (SEQ ID NO:3), or an analog thereof, wherein X is any amino acid; Z₁-Z₄ are each any amino acid, so long as at least one of Z₁-Z₄ is W, and the remaining of Z₁-Z₄ include one D, two D's, one E, two E's, or one D and one E; m and n are independently 0-3, but n+m must be two or greater; and o and p are independently 0-2. The peptide may be bound to a support, such as a bead, a membrane, a wafer, a bag, a filament, a dipstick, a microtiter plate or well thereof, or a chip. The peptide may comprise a capture agent that permits binding of the capture agent to a support.

Yet additional embodiment comprises a method of detecting a natural antibody in an antibody-containing fluid sample comprising (a) contacting the sample with at least one peptide selected from the group consisting XL(X)ₘ, (X)ₘTP(X)ₙ (SEQ ID NO:1), (X)ₒY₁Y₂Y₃Y₄Y₅(X)ₚ (SEQ ID NO:2) and (X)ₒZ₁Z₂Z₃Z₄(X)ₚ (SEQ ID NO:3), or an analog thereof, wherein X is any amino acid; Z₁-Z₄ are each any amino acid, so long as at least one of Z₁-Z₄ is W, and the remaining of Z₁-Z₄ include one D, two D's, one E, two E's, or one D and one E; m and n are independently 0-3, but n+m must be two or greater; and o and p are independently 0-2, and (b) detecting natural antibody bound to the peptide. The may comprise detecting natural antibody bound to a support surface, wherein the support surface has the peptide bound thereto. Detecting may comprise binding of a labeled anti-Ig antibody to the bound natural antibody. The anti-Ig antibody may be labeled with an enzyme or a calorimetric, fluorescent or bioluminescent label. Detecting the natural antibody may be diagnostic of an autoimmune disease in the subject from which the sample was obtained.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D—Binding of polyspecific IgM to screening Ags and phage-displayed peptides in ELISA. (FIGS. 1A-C) Solid lines with symbols: IgM binding to common screening Ags. Dashed lines with symbols: IgM binding to cognate phage-displayed peptides. Dashed lines without symbols: IgM binding to single-stranded DNA and double-stranded. (FIG. 1D) Activation of complement by IgM (2 μg/ml) bound to peptides carrying consensus determinants. All incubations were done in triplicate. Data are shown as mean±S.D. and are representative of at least three independent experiments.

FIGS. 2A-F—IgM-binding competition between synthetic and phage-displayed peptides (FIGS. 2A-C) and between synthetic peptides and screening Ags (FIG. 2D-D). Polyspecific IgM (2 μg/ml) was incubated with immobilized Ags in the presence of synthetic peptides in TBS/1% BSA/0.05% Tween-20 and bound IgM was detected by ELISA. Dashed lines-IgM binding to phage-displayed peptides devoid of consensus determinants. Data are representative of at least three independent experiments.

FIGS. 3A-C—IgM binding to phage-displayed peptides and screening Ags in 95-97% FBS_{EDTA}. Polyspecific IgM was diluted with fetal bovine serum/5 mM EDTA to a final concentration of 2 μg/ml and immediately incubated with immobilized Ags. Bound IgM was detected by ELISA. All incubations were done in triplicate. Data are shown as mean±S.D. and are representative of at least three independent experiments.

FIGS. 4A-I—IgM binding to phage-displayed peptides and screening Ags at different concentrations of fetal bovine serum/5 mM EDTA, heat-inactivated fetal bovine serum or mouse Rag-1 serum. Polyspecific IgM (2 μg/ml) was incubated with immobilized Ags in serum that was serially diluted with TBS/1% BSA/0.05% Tween-20 and bound IgM was detected by ELISA. Dashed lines-IgM binding to cognate peptides devoid of consensus determinants. Data are representative of at least three independent experiments.

FIGS. 5A-C—IgM binding to screening Ags in 91-99% heat-inactivated fetal bovine serum at different IgM concentrations. Polyspecific IgM was diluted with FBS^{HI} and immediately incubated with immobilized Ags. Bound IgM was detected by ELISA. All incubations were done in triplicate. Data are shown as mean±S.D. and are representative of at least three independent experiments.

FIGS. 6A-L—IgM binding to screening Ags in fractionated and in extensively dialyzed fetal bovine serum. Polyspecific IgM (2 μg/ml) was incubated with immobilized Ags in FBS-P (45% ammonium sulfate-precipitated fraction), or FBS-S (45% ammonium sulfate supernatant) or control non-fractionated FBS and bound IgM was detected by ELISA (FIGS. 6A-I). IgM incubations in heat-inactivated fetal bovine serum dialyzed for 48 h against four changes of TBS was conducted in the same fashion (FIGS. 6J-L). All incubations were done in triplicate. Data are shown as mean±S.D. and are representative of two independent experiments.

FIGS. 7A-F—IgM binding to screening Ags in DMEM. Polyspecific IgM (2 μg/ml) was incubated with immobilized Ags in DMEM and bound IgM was detected by ELISA. All incubations were done in triplicate. Data are shown as mean±S.D. and are representative of at least three independent experiments.

FIGS. 8A-P—IgM binding to muscle tissue sections in TBS/1% BSA and 98% heat-inactivated fetal bovine serum. Polyspecific IgM was diluted with TBS/1% BSA or heat-inactivated fetal bovine serum to a final concentration of 2 μg/ml and immediately incubated with non-fixed muscle tissue sections. After incubation, sections were fixed and bound IgM was stained and detected by confocal microscopy. Unless indicated otherwise, images are shown as projections of nine stacked optical sections with a thickness of 0.4 μm each. In each image pair, one image is stained for IgM only (red) and the other one is stained for IgM (red), actin (green), and nuclei (blue). (FIG. 8A) and (FIG. 8B) IgM41 in TBS/1% BSA/0.05% Tween-20; (FIG. 8C) and (FIG. 8D) IgM41 in heat-inactivated fetal bovine serum; (FIG. 8E) and (FIG. 8F) IgM58 in TBS/1% BSA/0.05% Tween-20; (FIG. 8G) and (FIG. 8H) IgM58 in heat-inactivated fetal bovine serum; (FIG. 81) and (FIG. 8J) IgM60 in TBS/1% BSA/0.05% Tween-20; (FIG. 8K) and (FIG. 8L) IgM60 in heat-inactivated fetal bovine serum; (FIG. 8M) and (FIG. 8N) IgM41 in heat-inactivated fetal bovine serum, individual optical sections (0.4 μm); (FIG. 80) and (FIG. 8P) control samples without IgM.

### DESCRIPTION OF ILLUSTRATIVE EMBODIMENTS

IgM molecules are highly sensitive to “molecular crowding” conditions due to their large size and disk-like shape (Ellis, 2001). Therefore, even weak reactivity of surface Ags with plasma macromolecules could potentially create a “crowded” environment that would restrict IgM penetration (Sebestyen et al., 2006). To examine the relationship between the medium composition and polyreactivity, the inventors compared the binding specificities expressed by polyspecific IgM Abs in conventional buffer media and undiluted sera deficient in immunoglobulins. The crucial element of this study was the use of small peptide Ags that were recognized by tested IgM along with conventional screening Ags. The inventors have shown previously that the vast majority of random C-terminal peptides displayed on T7 phage react with natural IgM Abs present in undiluted serum and blood (Sokoloff et al., 2000; 2001; 2004). The IgM recognition of these peptides is strikingly specific (Sokoloff et al., 2000; 2001; 2004). Based on these observations, the inventors set out to select analogous peptide Ags recognized by polyspecific IgM and use such medium-independent Ags as “standards” in testing IgM binding to common screening Ags.

This study demonstrates that natural IgM Abs acquire narrow binding specificity when transferred from buffer into undiluted serum. It appears that different Abs may lose polyspecificity via different mechanisms. The molecular mechanisms that mediate the loss of IgM polyspecificity are not known. The inventors speculate that there could be three general mechanisms contributing to this process. First, natural IgM Abs could react with serum “cofactors,” which would reduce their conformational plasticity and, thereby, polyspecificity (Foote and Milstein, 1994; James et al., 2003; Tissot et al., 2002). Second, cysteine-rich IgM molecules could undergo conformational changes in response to variations in the medium redox potential (McIntyre, 2004). Third, serum macromolecules reacting with immobilized Ags could create a “crowded” environment with restricted IgM penetration (Ellis, 2001; Sebestyen et al., 2006), which would be similar to the exclusion of large proteins from a glycosylated cell surface (Owen and Campbell, 1998). The SDS-PAGE of FBS proteins binding to immobilized NP-BSA, NIP-BSA, Flu-BSA and thyroglobulin, with BSA as a negative control, has shown enrichment for β2-macroglobulin (β2-M; data not shown). α2-M is a bulky protein (˜17 nm) that binds a variety of ligands (Armstrong and Quigley, 1999) and seems to be well suited for inhibiting IgM-binding through a steric repulsion mechanism. Further studies, exploring the ability of β2-M to affect natural IgM binding, are under way. The serum-induced loss of polyspecificity suggests that the natural IgM Abs showing broad polyspecificity in vitro may express narrow specificity in vivo. This might explain why many endogenous Ags are comparably reactive with autologous natural Abs in vitro while just few of them effectively elicit autoimmune responses in vivo (Shan et al., 1994). The loss of IgM polyspecificity in serum is consistent with the inventors' observations that the IgM present in normal undiluted serum contains a large variety of peptide-specific binding activities but no significant polyspecific activity directed against the same peptides (Sokoloff et al., 2000; 2001; 2004).

The inventors found that the presence of undiluted serum led to the acquisition of virtually monoreactive properties by polyspecific IgM Abs. In addition, they identified peptide mimitopes (peptides that mimic epitopes) that are specifically recognized by the majority of Nabs in undiluted serum. Most of these mimitopes contain a W residue and 1 or 2 D/E residues (W-determinant) and are recognized by all tested monoclonal antibodies, normal serum IgM, and both spleen and peritoneal B-cells. The universal nature of this mimotope is illustrated by the observations that the amount of normal mouse serum IgM bound to immobilized phage displaying W determinants is approximately the same as the amount of IgM bound to immobilized anti-IgM antibody over the whole range of serum dilution. Other determinants recognized by NAbs include the dipeptide TP (TP-determinant) and the peptide DGAXLXXX (L-determinant), where X is any amino acid residue. Synthetic peptides that mimic W-, TP- and L-determinants suppress the binding of NAbs to these determinants in vitro. Indole carboxylic acids that mimic W-determinants, particularly indole-3-propionic acid (IPA), suppress the binding of Nabs to W-determinants in vitro as well. IPA also suppresses the B-cell secretion of Nabs in ELISPOT experiments. IPA added to drinking water prevents the development of lymphoproliferation in autoimmune MRL/1pr mice, as evidenced by the absence of lymph node and spleen enlargement. IPA also prevents the appearance in adult MRL/1pr mice of IgG antibodies reacting with W determinants. This suggests that IPA prevents natural IgM antibodies from isotype switching, which has been implicated in the development of pathogenicity. Along with IPA, indole-3-acetic acid, indole-3-acrylic acid, indole-3-butiric acid and N-acetyl-tryptophan (net negative charge) also inhibited IgM binding to W-determinants. In contrast, non-acetylated tryptophan (net neutral charge) had no effect. This suggests that a minimal W-determinant consists of an indole group and a negatively charged group connected through a spacer of variable length. The spacer “rigidity” may also vary. Thus, trans-3-indoleacrylic acid, with a spacer rendered “rigid” by its double bond, inhibited IgM binding to W-determinates at least as effectively as IPA. The inhibition of IgM binding to W determinants was also observed with N-(indolylacetyl)-L-Ala, N-(indolylacetyl)-DL-Asp and N-(indolylacetyl)-L-Ile. In this compounds, an amino acid residue was a donor of a free carnoxyl group. 2-Benzimidazolepropionic acid also inhibited IgM binding to W determinants to a significant degree, indicating that the indole group and the orientation of the negatively charged group relative to the indole group can be modified.

Thus, it is proposed that the W-determinants and their synthetic analogs of both peptide and non-peptide origin can be used (i) as therapeutic agents that selectively or globally suppress or modulate the activity and survival of B-cells both in vitro and in vivo; (ii) as immobilized ligands that selectively remove undesirable immunoglobulins from diseased plasma or blood; (iii) as detection agents in measuring the functional activity of B-cells in health and disease; and (iv) as detection agents in measuring the total amount of natural antibodies of different isotypes in serum, plasma or blood. TP- and L-determinants can also be used in the same way.

### 1. Peptides or Polypeptides

The present invention relates to the use of various peptides that represent peptide “mimitope” for natural antibodies. As discussed above, Most of these mimitopes contain a W residue and 1 or 2 D/E residues (W-determinant), and other determinants include the dipeptide TP (TP-determinant) and the peptide DGAXLXXX (L-determinant), where X is any amino acid residue.

A. Structural Features

The peptides of the present invention may be defined structurally, in certain aspects, as follows:

DGAXL(X)ₘ, (X)ₘTP(X)ₙ and (X)ₒZ₁Z₂Z₃Z₄(X)ₚ, or an analog thereof, wherein:
X is any amino acid;
Z₁-Z₄ are each any amino acid, so long as at least one of Z₁-Z₄ is W, and the remaining of Z₁-Z₄ include one D, two D's, one E, two E's, or one D and one E;
m and n are independently 0-3, but n+m must be two or greater; and
o and p are independently 0-2.
   Peptides of the present invention will comprise molecules of 4 to about 50 residues in length, may be less than 35 residues, less than 30 residues, less than 25 residues, less than 20 residues, less than 15 residues, less than 13 residues, or less than 11 residues, including 4, 5, 6, 7, 8, 9, 10, 11, 12, 13, 14, 15, 16, 17, 18, 19 or 20 residues. The peptides may be generated synthetically or by recombinant techniques, and are purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

The peptides may be labeled using various molecules, such as fluorescent, chromogenic or colorimetric agents. The peptides may also be linked to other molecules, including other anti-pain agents. The links may be direct or through distinct linker molecules. The linker molecules in turn may be subject, in vivo, to cleavage, thereby releasing the agent from the peptide. Peptides may also be rendered multimeric by linking to larger, and possibly inert, carrier molecules.

B. Peptide Analogs

The present invention may employ peptides that comprise modified, non-natural and/or unusual amino acids. A chart of exemplary, but not limiting, modified, non-natural and/or unusual amino acids is provided herein below. Chemical synthesis may be employed to incorporate such amino acids into the peptides of interest.

### Modified, Non-Natural and Unusual Amino Acids

| Abbr. | Amino Acid |
|---|---|
| Aad | 2-Aminoadipic acid |
| BAad | 3-Aminoadipic acid |
| BAla | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| BAib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| Aile | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In addition to the variants discussed above, the present inventors also contemplate that structurally similar compounds may be formulated to mimic the key portions of peptide or polypeptides of the present invention. Such compounds, which may be termed “peptidomimetics,” may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al., 1998). A structural motif that appears to be evolutionarily conserved in certain toxins is small (30-40 amino acids), stable, and high permissive for mutation. This motif is composed of a beta sheet and an alpha helix bridged in the interior core by three disulfides.

β II turns have been mimicked successfully using cyclic L-pentapeptides and those with D-amino acids (Weisshoff et al., 1999). Also, Johannesson et al. (1999) report on bicyclic tripeptides with reverse turn inducing properties.

Methods for generating specific structures have been disclosed in the art. For example, α-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

C. Fusion Proteins

Another variant is a fusion protein. This molecule generally has all or a substantial portion of the original molecule, in this case a peptide of the present invention, linked at the N- or C-terminus, to all or a portion of a second peptide or polypeptide. For example, fusions may employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

D. Purification of Peptides

It may be desirable to purify peptides, variants, peptide-mimics or analogs thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

E. Peptide Synthesis

Peptides may be generated synthetically for use in various embodiments of the present invention. Because of their relatively small size, the peptides of the invention can be synthesized in solution or on a solid support in accordance with conventional techniques. Various automatic synthesizers are commercially available and can be used in accordance with known protocols. See, for example, Stewart & Young, (1984); Tam et al., (1983); Merrifield, (1986); Barany and Merrifield (1979), each incorporated herein by reference. Short peptide sequences, or libraries of overlapping peptides, usually from about 4 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

### 2. Nucleic Acids

Another aspect of the present invention concerns isolated DNA segments and recombinant vectors encoding peptides of the present invention, the creation and use of recombinant host cells through the application of DNA technology, that express such peptides.

It will also be understood that nucleic acid sequences may include additional sequences encoding additional N- or C-terminal amino acids, and yet still be essentially as set forth in one of the sequences disclosed herein, so long as the sequence meets the criteria set forth above, including the maintenance of biological protein, polypeptide or peptide activity where an amino acid sequence expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

In certain embodiments, the present invention involves either the production of peptides. Such methods both rely upon expression constructs containing peptide coding regions and the means for its expression, plus elements that permit replication of the constructs. A variety of elements and vector types are discussed below.

A. Selectable Markers

In certain embodiments of the invention, expression constructs of the present invention contain nucleic acid constructs whose expression may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as herpes simplex virus thymidine kinase (tk) may be employed. Immunologic markers also can be employed. The selectable marker employed is not believed to be important, so long as it is capable of being expressed simultaneously with the nucleic acid encoding a gene product. Further examples of selectable markers are well known to one of skill in the art and include reporters such as EGFP, β-gal or chloramphenicol acetyltransferase (CAT).

B. Polyadenylation Signals

One will typically desire to include a polyadenylation signal to effect proper polyadenylation of the transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

C. Control Regions

Throughout this application, the term “expression construct” is meant to include any type of genetic construct containing a nucleic acid coding for the peptide of interest. The nucleic acid encoding the peptide is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation.

For the purpose of recombinant production, prokaryotic (bacteria) and lower eukaryotic organisms (yeast) are preferred. Commercial vectors and expression systems, including appropriate host cells and methods for transformation and culture, are well known to those of skill in the art.

In other embodiments, promoters refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic.

In some circumstances, it may be desirable to regulate expression of a transgene in a vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly, tissue specific promoters may be used to effect transcription in specific tissues or cells so as to reduce potential toxicity or undesirable effects to non-targeted tissues. For example, promoters that are selectively active in B-cells may find particular utility in accordance with the present invention.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

D. Gene Transfer

In order to effect recombinant expression of peptides of the present invention, it is necessary to transfer the appropriate expression construct into a host cell of interest. Such transfer may employ viral or non-viral methods of gene transfer. This section provides a discussion of methods and compositions of gene transfer.

In one embodiment of the invention, an expression construct comprises a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses such as the papovaviruses (simian virus 40, bovine papilloma virus, and polyoma) (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. A wide variety of viruses are now used to successfully transfer genetic material to eukaryotic cells, including adenovirus, AAV, vaccinia virus (Ridgeway, 1988; Baichwal and Sugden, 1986; Coupar et al., 1988) canary pox virus, and herpesviruses may be employed.

Several non-viral methods for the transfer of expression constructs into cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

### 3. Screening Assays

The present invention also contemplates the screening of compounds, e.g., peptides, peptide-mimics, variants, analogs or small molecules, for various abilities to interact with NAbs and/or modulate B-cell function in vitro and/or in vivo. In the screening assays of the present invention, the candidate substance may first be screened for basic biochemical activity—e.g., binding to a target molecule (e.g., NAb)—and then tested for its ability to modulate B-cell function.

A. Modulators

The present invention provides methods of screening for agents that bind to Nabs. In an embodiment, the present invention is directed to a method of:
(a) providing a Nab under conditions that replicate physiologic binding conditions;
(b) contacting the Nab with a candidate peptide or peptide analog; and
(c) determining the binding of the candidate to the Nab,
   wherein binding to the Nab identifies the candidate as a Nab mimitope. Measuring binding may be direct, by identifying a candidate-Nab complex, by identifying labeled candidate associated with the Nab, or by assessing the inhibition of binding of a known mimitope to a Nab. In still yet other embodiments, one would look at the effect of a candidate on B-cell function.

As used herein, the term “candidate substance” refers to any molecule that may potentially bind to a Nab, but typically will refer to a peptide or peptide analog. New candidates may be modeled off known mimitopes using “rational drug design,” and include making predictions relating to the structure of target molecules. The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules.

On the other hand, one may simply acquire, from various commercial sources, small molecule, peptide or peptoid libraries that are believed to meet the basic criteria for useful drugs in an effort to “brute force” the identification of useful compounds. Screening of such libraries is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

It will, of course, be understood that all the screening methods of the present invention are useful in themselves notwithstanding the fact that effective candidates may not be found. The invention provides methods for screening for such candidates, not solely methods of finding them.

B. In vitro Assays

A quick, inexpensive and easy assay to run is an antibody binding assay. Binding of a molecule to a Nab is likely to be inhibitor in and of itself due to steric, allosteric or charge-charge interactions. Such assays can be performed in solution or on a solid phase and can be utilized as a first round screen to rapidly eliminate certain compounds before moving into more sophisticated screening assays. In the context of the present invention, the use of a physiologic or semi-physiologic environment, e.g., undiluted serum, is specifically contemplated.

The target (e.g., Nab) may be either free in solution, fixed to a support, expressed in or on the surface of a cell. Either the target or the candidate may be labeled, thereby permitting determination of binding. Competitive binding assays can be performed in which a known mimitope is used. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The test compounds are reacted with the target and washed. Bound test compounds may be detected by various methods.

C. In cyto Assays

Various cells that express Nabs, in particular B-cells, can be utilized for screening of candidate substances. Depending on the assay, culture may be required. Labeled candidate substances are contacted with the cell and binding assessed. Various readouts for binding of candidate substances to cells may be utilized, including fluorescent microscopy and FACS.

Alternatively, functional assays may look at whether B-cell activity can be impacted by a candidate peptide or peptide analog. Activity can be B-cell proliferation or antibody secretion.

D. In vivo Assays

The present invention particularly contemplates the use of various animal models. For example, an autoimmune mouse can be used to assess the impact of candidate substance in vivo. Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by oral, sublingual, intratracheal instillation, bronchial instillation, intradermal, subcutaneous, intramuscular, intraperitoneal or intravenous injection. Specifically contemplated are oral administration and systemic intravenous injection.

### 4. Modulating and Detecting Natural Antibodies

### A. Modulation of B-Cell NAb Production

The peptides and analogs of the present invention will find utility in the treatment of various forms of autoimmune disease. Applicants believe these of peptides and analogs thereof can inhibit the production of antibodies that would otherwise attack native antigens in a host, thereby causing various pathologic inflammatory responses. in accordance with the present invention may be

The following disease states are contemplated for treatment in accordance with the present invention.

i. Systemic Lupus Erythematosus

Systemic lupus erythematosus (SLE) is an autoimmune chronic inflammatory disease that most commonly affects the skin, joints, kidneys, heart, lungs, blood vessels, and brain. The most common symptoms include fatigue, muscle aches, low-grade fever, skin rashes, and kidney problems that are sometimes severe enough to require dialysis or transplant. Symptoms may also include a characteristic facial rash (“butterfly rash”), photosensitivity, and poor circulation to the extremities with cold exposure, known as Raynaud's phenomenon. Rheumatoid arthritis is another chronic autoimmune disease, and most people with SLE will develop arthritis during the course of their illness with similar symptoms to rheumatoid arthritis. Because SLE can affect the walls of the blood vessels, young women with SLE are at significantly higher risk for heart attacks from coronary artery disease. For many patients, alopecia occurs as SLE worsens.

Women who become pregnant with SLE are considered “high risk.” These women have an increased risk of miscarriages, and the incidence of flares can increase with pregnancy. Antibodies from SLE can be transferred to the fetus, resulting in “neonatal lupus.” Symptoms of neonatal lupus include anemia and skin rash, with congenital heart block being less common. Unlike SLE, neonatal lupus resolves after six months as the newborn metabolizes the mother's antibodies.

Because the symptoms of SLE can vary widely, accurate diagnosis is difficult. A diagnosis of SLE is suggested for a patient who meets four or more of the eleven criteria established by the American Rheumatism Association, but there is currently no single test that establishes the diagnosis of SLE. However, these criteria are not definitive. The criteria are based on the symptoms of SLE, but also include the presence of anti-DNA, antinuclear (ANA), or anti-Sm antibodies, a false positive test for syphilis, anticardiolipin antibodies, lupus anticoagulant, or positive LE prep test. Some patients are diagnosed with SLE who manifest fewer than four criteria, while other such patients remain undiagnosed.

Most people with SLE test positive for ANA. Even so, the test is not definitive, as a number of conditions can cause a positive ANA test. Other antibody tests that can aid in a diagnosis of SLE or other autoimmune conditions include anti-RNP, anti-Ro (SSA), and anti-La (SSB).

There is currently no cure for SLE, and the illness remains characterized by alternating periods of illness, or flares, and periods of wellness, or remission. The current goal of treatment is to relieve the symptoms of SLE, and to protect the organ systems affected by decreasing the level of autoimmune activity. More and better quality rest is prescribed for fatigue, along with exercise to maintain joint strength and range of motion. DHEA (dehydroepiandrosterone) can reduce fatigue and thinking problems associated with SLE. Physicians also commonly prescribe Nonsteroidal antiinflammatory drugs (NSAIDs) for pain and inflammation, although this can cause stomach pain and even ulcers in some patients.

Hydroxychloroquine, an anti-malarial medication, can be effective in treating fatigue related to SLE as well as skin and joint problems. Hydroxychloroquine also decreases the frequency of excessive blood clotting in some SLE patients. Corticosteroids are needed for more serious cases, although the serious side effects, such as weight gain, loss of bone mass, infection, and diabetes limits the length of time and dosages at which they can be prescribed. Immunosuppressants, or cytotoxic drugs, are used to treat severe cases of SLE, but again serious side effects such as increased risk of infection from decreased blood cell counts are common.

Possible future therapies include stem cell transplants to replace damaged immune cells and radical treatments that would temporarily kill all immune system cells. Other future treatments may include “biologic agents” such as the genetically engineered antibody rituximab (anti-CD20) that block parts of the immune system, such as B cells. Recently, two groups of researchers found that even partial restoration of function of an inhibitory Fc receptor prevented the development of SLE in several strains of mice that were genetically prone to the disease. Reviewed in Kuehn, Lupus (2005).

SLE is much more common among women than men, with women comprising approximately 90% of all SLE patients. It is also three times more common in African American women than in women of European descent, although the incidence is also higher among women of Japanese and Chinese ancestry.

Because widely varying symptoms of SLE make accurate diagnosis difficult, the exact number of people who suffer from SLE is unknown. The Lupus Foundation of America, however, estimates that approximately 1,500,000 Americans have some form of lupus. The prevalence of SLE is estimated to be about 40 per 100,000.

ii. Rheumatoid Arthritis

The exact etiology of RA remains unknown, but the first signs of joint disease appear in the synovial lining layer, with proliferation of synovial fibroblasts and their attachment to the articular surface at the joint margin (Lipsky, 1998). Subsequently, macrophages, T cells and other inflammatory cells are recruited into the joint, where they produce a number of mediators, including the cytokines interleukin-1 (IL-1), which contributes to the chronic sequelae leading to bone and cartilage destruction, and tumour necrosis factor (TNF-α), which plays a role in inflammation (Dinarello, 1998; Arend & Dayer, 1995; van den Berg, 2001). The concentration of IL-1 in plasma is significantly higher in patients with RA than in healthy individuals and, notably, plasma IL-1 levels correlate with RA disease activity (Eastgate et al., 1988). Moreover, synovial fluid levels of IL-1 are correlated with various radiographic and histologic features of RA (Kahle et al., 1992; Rooney et al., 1990).

In normal joints, the effects of these and other proinflammatory cytokines are balanced by a variety of anti-inflammatory cytokines and regulatory factors (Burger & Dayer, 1995). The significance of this cytokine balance is illustrated in juvenile RA patients, who have cyclical increases in fever throughout the day (Prieur et al., 1987). After each peak in fever, a factor that blocks the effects of IL-1 is found in serum and urine. This factor has been isolated, cloned and identified as IL-1 receptor antagonist (IL-1ra), a member of the IL-1 gene family (Hannum et al., 1990). IL-1ra, as its name indicates, is a natural receptor antagonist that competes with IL-1 for binding to type I IL-1 receptors and, as a result, blocks the effects of IL-1 (Arend et al., 1998). A 10- to 100-fold excess of IL-1ra may be needed to block IL-1 effectively; however, synovial cells isolated from patients with RA do not appear to produce enough IL-1ra to counteract the effects of IL-1 (Firestein et al., 1994; Fujikawa et al., 1995).

iii. Sjögren's Syndrome

Primary Sjögren's syndrome (SS) is a chronic, slowly progressive, systemic autoimmune disease, which affects predominantly middle-aged women (female-to-male ratio 9:1), although it can be seen in all ages including childhood (Jonsson et al., 2002). It is characterized by lymphocytic infiltration and destruction of the exocrine glands, which are infiltrated by mononuclear cells including CD4+, CD8+ lymphocytes and B-cells (Jonsson et al., 2002). In addition, extraglandular (systemic) manifestations are seen in one-third of patients (Jonsson et al., 2001).

The glandular lymphocytic infiltration is a progressive feature (Jonsson et al., 1993), which, when extensive, may replace large portions of the organs. Interestingly, the glandular infiltrates in some patients closely resemble ectopic lymphoid microstructures in the salivary glands (denoted as ectopic germinal centers) (Salomonsson et al., 2002; Xanthou & Polihronis, 2001). In SS, ectopic GCs are defined as T and B cell aggregates of proliferating cells with a network of follicular dendritic cells and activated endothelial cells. These GC-like structures formed within the target tissue also portray functional properties with production of autoantibodies (anti-Ro/SSA and anti-La/SSB) (Salomonsson &, Jonsson, 2003).

In other systemic autoimmune diseases, such as RA, factors critical for ectopic GCs have been identified. Rheumatoid synovial tissues with GCs were shown to produce chemokines CXCL13, CCL21 and lymphotoxin (LT)-13-β (detected on follicular center and mantle zone B cells). Multivariate regression analysis of these analytes identified CXCL13 and LT-β as the solitary cytokines predicting GCs in rheumatoid synovitis (Weyand & Goronzy, 2003). Recently CXCL13 and CXCR5 in salivary glands has been shown to play an essential role in the inflammatory process by recruiting B and T cells, therefore contributing to lymphoid neogenesis and ectopic GC formation in SS (Salomonsson et al., 2002).

iv. Other Autoimmune Diseases

The following autoimmune diseases or other diseases with an autoimmune component may be subject to treatment according to the present invention: chronic leukocytic leukemia, juvenile onset diabetes mellitus, Wegener's granulomatosis, inflammatory bowel disease, polymyositis, dermatomyositis, multiple endocrine failure, Schmidt's syndrome, autoimmune uveitis, Addison's disease, adrenalitis, Graves' disease, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, multiple sclerosis, subacute cutaneous lupus erythematosus, hypoparathyroidism, Dressler's syndrome, myasthenia gravis, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyl), and telangiectasia), adult onset diabetes mellitus (Type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, glomerulonephritis, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and Non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, and autoimmune gonadal failure.

### B. Combined Therapy

In order to increase the effectiveness of peptides or mimics or analogs thereof, it may be desirable to combine these compositions with another agent effective in the treatment of autoimmune disease. The terms “contacted” and “exposed,” when applied to a cell, tissue or organism, are used herein to describe the process by which a peptide or mimic or analog and/or another agent are delivered to a target cell, tissue or organism or are placed in direct juxtaposition with the target cell, tissue or organism.

The peptide or mimic or analog may precede, be co-current with and/or follow the other agent(s) by intervals ranging from minutes to weeks. In embodiments where the peptide or mimic or analog, and other agent(s) are applied separately to a cell, tissue or organism, one would generally ensure that a significant period of time did not expire between the time of each delivery, such that the peptide and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the peptide or mimic or analog. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 18 hours, about 24 hours, about 36 hours, about 48 hours, about 3 days, about 4 days, about 5 days, about 6 days, about 7 days, about 8 days, about 9 days, about 10 days, about 11 days, about 12 days, about 13 days, about 14 days, about 21 days, about 4 weeks, about 5 weeks, about 6 weeks, about 7 week or about 8 weeks or more, and any range derivable therein, prior to and/or after administering the peptide or mimic or analog.

Various combination regimens of the treatment and one or more other anti-pain agents may be employed. Non-limiting examples of such combinations are shown below, wherein a peptide/peptide analog composition is “A” and the anti-inflammatory/immunosuppressive agent is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the peptide/peptide analog to a cell, tissue or organism may follow general protocols for the administration of pharmaceuticals, taking into account the toxicity, if any. It is expected that the treatment cycles would be repeated as necessary. In particular embodiments, it is contemplated that various additional agents may be applied in any combination with the present invention. Agents used to treat autoimmune disease, which may be used in combination therapies, include anti-inflammatory agents such as steroid, NSAIDs, peptides, antibodies, cytokines, soluble cytokine receptors, and immunosuppressive agents.

### C. Purifying/Isolating NAbs

In another embodiment, methods of separating NAbs from plasma, both to prepare reagents for laboratory use and to treat disease states characterized by an overproduction of such antibodies, are contemplated. In order to perform such purification, one will provide a support onto which peptides or peptidomimetics of the present invention are disposed. By passing plasma across this support under conditions permitting binding, one can remove Nabs from the plasma, which can then be returned to a patient if appropriate care is taken to maintain the sterility of the plasma. In this way, Nabs may be removed from the plasma, and their harmful effects diminished within the subject.

### D. Detecting NAbs

In another aspect of the invention, the inventors propose that peptides and peptide analogs as described herein can be used diagnostically to monitor the autoimmune status of a subject. Increasing levels of Nabs indicate that a subject is generating an immune response against self-determinants, and thus at risk for autoimmune diseases, or that naïve B cells accumulate at pathological levels, as observed in chronic leukocytic leukemia and Waldenstrom's macroglobulemia for instance. This approach is possible since the W determinant is recognized by the majority of otherwise unrelated Nab.

The assays will generally take the form of a classic antigen-antibody-antibody “sandwich” assay in which the peptide or peptide analog of the present invention is fixed to a support—a bead, microtiter well, column or filter—and then a body fluid (e.g., serum) is passed over the support under conditions permitting binding of appropriate antibodies to peptides or analogs. The support is then washed to remove non-specifically bound proteins, and the presence of bound Nab is detected by use of a secondary anti-Ig antibody, often labeled with a detectable moiety such a radiolabel, a fluorescent label, a chemilluminescent label or an enzyme. Alternatively, the binding will be detected due to physical changes in the Ag support, as illustrated by the Biacore technique. The presence and quantity of Nab can be ascertained and a diagnostic or prognostic evaluation made.

### E. Pharmaceutical Formulations

Pharmaceutical formulations of the present invention comprise an effective amount of a peptide or peptide analog dissolved or dispersed in a pharmaceutically acceptable carrier. The phrases “pharmaceutical or pharmacologically acceptable” refer to compositions that do not produce an adverse, allergic or other untoward reaction when administered to an animal, such as, for example, a human, as appropriate. The preparation of such pharmaceutical compositions are known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with the active ingredient, its use in the therapeutic or pharmaceutical compositions is contemplated.

The pharmaceuticals of the present invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intramuscularly, intraperitoneally, subcutaneously, subconjunctival, intravesicularlly, mucosally, intrapericardially, intraumbilically, intraocularally, orally, topically, locally, inhalation (e.g., aerosol), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The pharmaceuticals may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain embodiments, the compositions are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### 5. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Antibodies and antigens. AP— and biotin-conjugated goat IgG specific for the mouse IgM μ-chain and rat Abs (clones EM34.1 and 9A8) specific for the mouse κ- and λ-chains (Sigma, St. Louis, Mo.); C3 goat antiserum (Bethyl Laboratories, Montgomery, Tex.); goat IgG specific for the IgM Fc5μ-region and rabbit IgG directed against the goat IgG γ-chain (Pierce, Rockford, Ill.); Fab Abs specific for the mouse Fab region (Jackson ImmunoRe search, West Grove, Pa.); anti-phage T7 rabbit IgG (Sokoloff et al., 2004); Flu(6)-BSA, TNP(14)-BSA, TNP(15)-BSA, NP(24)-BSA, NIP(31)-BSA, DNP(10)-BSA and PC(15)-BSA (Biosearch Technologies, Novato, Calif.); rabbit muscle myosin, bovine thyroglobulin and calf thymus ssDNA and dsDNA (Sigma, St. Louis, Mo.); blocking BSA (Pierce, Rockford, Ill.). Synthetic peptides Ac-PEGWN, Ac-RLTPR, Ac-DLLDR Ac-DGA-DLLDR (purity >98%, where the residues required for IgM recognition are underlined, were prepared at Mirus Corporation (Madison, Wis.).

Animals. Male Balb/c mice, 6-8 weeks old, were from Jackson (Bar Harbor, Minn.). Rag-1 mice (Jackson) were bred in our SPF facility. All animal work was conducted according to protocols approved by the Animal Care and Use Committee of The University of Wisconsin.

Sera. Undiluted FBS (HyClone, Logan, Utah) was heated at 56° C. for 1 h (FBS^{HI}) or supplemented prior to use with 5 mM EDTA (FBS^{EDTA}) to eliminate its residual complement activity. FBS contained ˜50 times less IgG (˜200 μg/ml) than adult bovine serum. With the typical IgG:IgM:IgA ratio for FBS being ˜3:1:1 (Ellis et al., 1978), this serum also was expected to contain small amounts of IgM and IgA. The FBS^{HI} precipitate (FBS-P) and supernatant (FBS-S) fractions were prepared by precipitation with 45% ammonium sulfate and dialyzed against TBS. Extensively dialyzed FBS^{HI} was prepared by dialysis against four changes of TBS over 48 h. Complement-grade Rag-1 mouse serum was prepared by clotting blood on ice for 1 h. Rag-1 serum used as a binding medium was prepared by clotting blood at room temperature for 2-4 h. Prior to use, this serum was supplemented with 5 mM EDTA. All FBS and Rag-1 sera were stored at −20° C. and −80° C., respectively.

Phage. A peptide library with the general structure DGA(X)₅, where DGA is a “spacer” and (X)₅ is a random sequence, was displayed at the C-terminus of the T7 phage protein 10B as described previously (Sokoloff et al., 2000; 2004). Phage was stored in TBS/1 mM MgCl₂/30% glycerol at −80° C.

Polyspecific IgM Abs. Hybridoma clones secreting IgM41, IgM58 and IgM60 were prepared from the splenocytes of naive 6-7-week-old Balb/c mice using the myeloma line NS1 as the fusion partner at Harlan Inc. (Madison, Wis.). Primary polyspecific clones (HAT medium) were detected by testing culture supernatants for reactivity with common screening Ags (Section 2.1) by ELISA (Section 2.7) and then subjected to several additional rounds of cloning using the limited dilution method (world-wide-web at hbps.com) and expanded in ascites. IgM was isolated by chromatography on a mannan-binding protein column using an ImmunoPure® IgM Purification Kit (Pierce, Rockford, Ill.). This procedure is conducted at neutral pH and does not involve the use of chaotropic or acidic agents (Nevens et al., 1992). Isolated IgM was dialyzed against TBS and stored at 0.4-1.5 mg/ml at −80° C. in aliquots. Unfractionated IgM was prepared by growing hybridomas in DMEM supplemented with 10% FBS^{HI}, 20 mM Hepes, 50 μM 2-ME, 1 mM Na-pyruvate, and 25 μg/ml gentamycin. Culture supernatants were filtered and stored at −80° C. in aliquots. The IgM concentration was measured by capture ELISA using MOPC 104E as a standard.

All three IgM's contained K-chains, as determined by ELISA using IgM_{λ} (MOPC 104E) and IgM_{κ} (TEPC 183) as standards. The V_{H} genes encoding for IgM41, IgM58 and IgM60 were sequenced (Seidl et al., 1997; GenBank accession nos. DQ340230, DQ340231, and DQ340232, respectively) and found to be identical to the germline genes V_{H}1/H13-3, V_{H}5/7183.9, and V_{H}1/J558.5 (GenBank accession no. X02459; AF290971, and AF303836, respectively). The V_{H} germline structure suggested that all three IgM's were typical representatives of polyspecific natural Abs (Casali and Schettino, 1996).

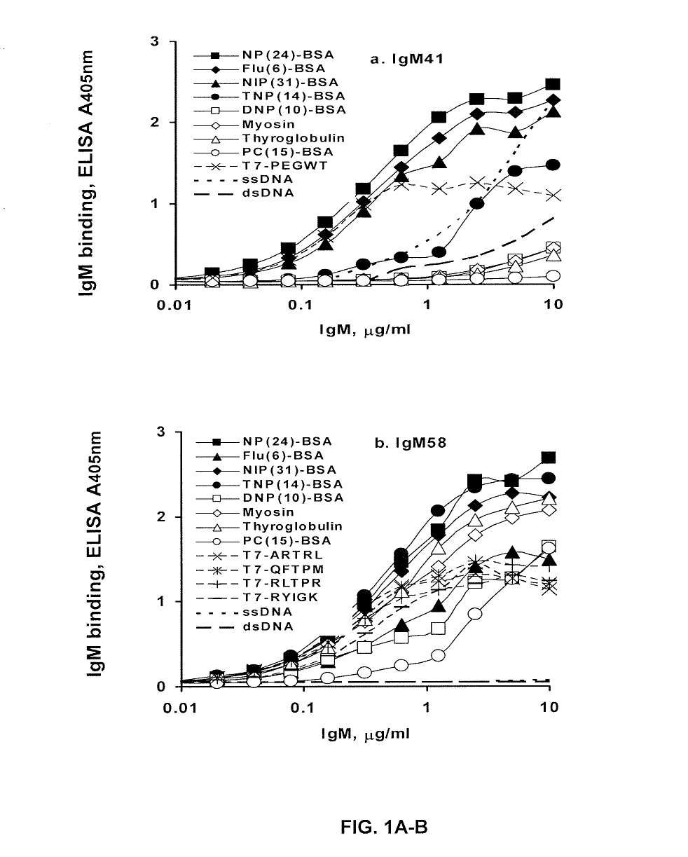

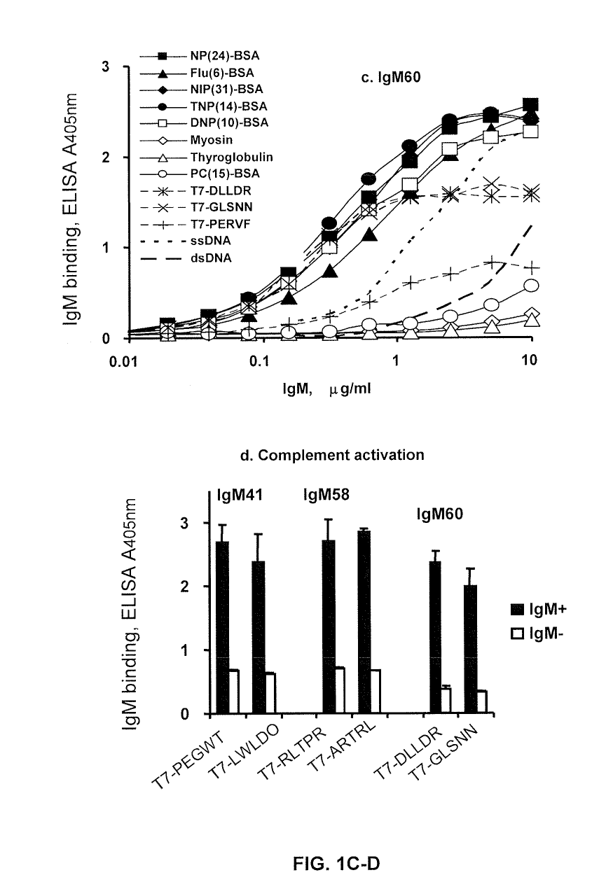

IgM41, IgM58 and IgM60 were chosen for this study due to different specificities that they expressed in reactions with common screening Ags. IgM41 and IgM60 demonstrated distinct preferences for hapten conjugates while similarly binding to ssDNA and dsDNA and not binding to myosin or thyroglobulin (FIGS. 1A and 1C). IgM58 reacted with myosin, thyroglobulin and various BSA-hapten conjugates while showing little reactivity with DNA (FIG. 1B).

Peptide selections. IgM denaturation on polystyrene (Schwab and Bosshard, 1992) was prevented by using ELISA plates coated with Protein G (Pierce, Rockford, Ill.). A high density of displayed peptides, 415 copies per phage particle, allowed selection of high-avidity IgM-phage complexes that were mediated by low-affinity IgM-peptide interactions (Perkins et al., 1991; Sokoloff et al., 2000; 2001). Non-specific IgM binding was minimized using displayed peptides that were just five amino acid residues long. The peptide length was sufficient, however, for specific IgM-peptide interactions (Sokoloff et al., 2001; 2004).

Plates were coated overnight with goat IgG specific for the IgM Fc5μ region (10 μg/ml; 50 μl/well) and then washed with TBS and coated with polyspecific IgM (2-50 μg/ml) in TBS/1% BSA/0.05% (TBS-BT) or DMEM/10% FBS. IgM-coated plates were washed and incubated with T7 peptide library (50 μl; 2×10¹¹ pfu/ml) in TBS-BT for 2 h. TBS-BT closely resembled typical binding media used in polyspecificity studies (Dighiero et al., 1983; Haspel et al., 1983). Unbound phage was washed out and bound phage was extracted with TBS/1% SDS for 30 min and amplified (Sokoloff et al., 2000). Two more selection rounds were conducted in the same manner. The structure of selected peptides was determined by DNA sequencing (Sokoloff et al., 2004). Consensus peptide residues are shown underlined throughout the text. Phage-displayed peptides are shown as T7-XXXXX, to distinguish them from synthetic peptides (Ac-XXXXX).

The selection system was tested with the monoclonal IgM produced by MOPC 104E (IgM104). Due to its high specificity for dextran 1→3, IgM104 was likely to react mostly with structurally-related peptide mimotopes (Schepers et al., 1978). The percentage of the input phage that bound to IgM104 increased from 0.007% in the 1st selection round to 1.6% and 13.2% in the 2nd and 3rd rounds, respectively. Seven of 10 selected peptides displayed the consensus sequence T7-A/G/N/K)LWKS (Table 1 a; IgM104), and the remaining peptides contained different elements of this sequence (Table 1B; IgM104). The absence of unrelated selected peptides suggested that the selection system could be used with polyspecific IgM Abs, which could potentially react with multiple unrelated mimotopes (Manivel et al., 2002).

IgM binding to various immobilized antigens was performed at 22-24° C. unless indicated otherwise. ELISA plates were coated overnight with phage (4×10¹⁰ pfu/ml), BSA-hapten conjugates (10 μg/ml) or purified proteins (10 μg/ml) in TBS (50 μl/well). The efficiency of phage immobilization, assessed with anti-T7 Ab, was similar for different clones (Sokoloff et al., 2004). IgM-binding was detected using an ELISA described previously (Sokoloff et al., 2004). IgM-binding to DNA was assessed in a similar fashion, using poly-d-lysine plates (Sigma, St. Louis, Mo.) coated with DNA (50 μg/ml) for 2 h and blocked with TBS/1% BSA (TBS-B). To measure the deposition of complement C3, IgM-phage complexes were incubated with 1.25% complement-grade Rag-1 mouse serum in TBS-BT/1 mM MgCl₂/1 mM CaCl₂ at 37° C. for 1 h. Plates were washed with TBS, incubated for 1 h with C3 antiserum in TBS-BT and then washed again and incubated for 1 h with AP-conjugated rabbit Ab directed against goat IgG. All remaining steps were as above.

Confocal microscopy. Balb/c mice were euthanized and their upper and lower limb muscles were immediately removed and embedded in OCT compound (Sakura, Calif.). Five-micron muscle sections were prepared and air-dried at room temperature. Sections were washed twice in PBS and incubated with polyspecific IgM (2 μg/ml) in TBS-B or 98% FBS^{HI} in a moist chamber at room temperature for 1 h. Next, sections were washed three times with PBS, fixed with 4% formaldehyde for 15 min, washed twice with PBS, and blocked using an avidin/biotin blocking kit (Vector Laboratories, Burlingame, Calif.). Blocked sections were rinsed with PBS and incubated with biotinylated anti-IgM Ab in TBS-B for 30 min. Sections were then washed twice with PBS and incubated with Cy3-streptavidin (Molecular Probes, Eugene, Oreg.) in TBS-B for 20 min. Nuclei and actin were counterstained with ToPro-3 and Alexa-488-Phalloidin (Molecular Probes, Eugene, Oreg.), respectively. Stained sections were washed twice with PBS and examined in a Carl Zeiss LSM 510 confocal microscope.

ELISPOT. Spleen and peritoneal washout were prepared using 10-12 wk-old Balb/c mice and B cells were isolated using the StemCell Technologies protocol and reagents. Isolated B cells were suspended in 10% FBS/DMEM and incubated overnight in a 96-well polypropylene plate containing indolylpropionic acid (IPA) solution at 0.625 mM −2.5 mM at 37° C. in the presence of 5% CO₂. The cells were transferred into a phage- or anti-IgM antibody-coated ELISPOT plate (Millipore MltiScreenHTS), at 200-15000 cells per well, and incubated overnight at 37° C. in the presence of 5% CO₂. Coating phage and goat anti-mouse IgM antibody (positive control) were used at 10¹¹ pfu/ml and 10 μg/ml, respectively. Phage clones displaying W determinants and control peptids lacking Trp residues were used. Coating was allowed to proceed overnight with gentle rocking. Next day, plates were blocked, washed and seeded with cells. LPS-stimulated spleen and peritoneal B cells were prepared by incubating cells in 10% FBS/DMEM containing 10 μg/ml LPS for 24, 48 and 72 hours (10⁶ cells/ml). Stimulated cells were transferred into ELISPOT plates (200-15000 cells/wells). ELISPOT plates were processed as follows. After discarding the well media, plate were washed once with H₂O and then 6 times with 0.05% Tween-20 in TBS and incubated with biotin-labeled anti-mouse IgM antibody in 1% BSA/TBS. Plate were washed as above and incubated with Neutravidin-AP in TBS. Finally, plates were washed 3 times with 0.05% Tween-20/TBS, then 3 times with saline, and incubated with color development solution. Developed plates were washed with H₂O, air dried, and sent to ZellNet (Fort Lee, N.J.) for spot counting.

In vivo experiments. Female LRL/1rp mice were receiving either normal water or 20 mM IPA (pH ˜7) starting from the age of 5 weeks. Mice were killed at the age of 22 weeks and their lymph nodes, spleens and kidneys were weighed and subjected to histological analysis.

### Example 2

### Results

The majority of peptides selected for binding to polyspecific IgM share common structural determinants. The phage yields observed in selection experiments with polyspecific IgM41 and IgM58 were comparable to those observed with IgM104 (Section 2.6). IgM41 bound 0.03%, 5.6%, and 11.9% of the input phage in the 1^{st}, 2^{nd}, and 3^{rd} rounds of selection, respectively. The corresponding values for IgM58 were 0.08%, 3.8%, and 15.2%. IgM60 bound phage more efficiently, with the yields of 0.5%, 25.8%, and 33.9%.

Seventeen of 20 peptides selected with IgM41 featured a rare Wresidue (W-peptides) in a −2 through −5 position, and 12 of these peptides contained one or more D/E residues a maximum of one residue away from the W residue. The combination of W and D/E residues was termed a consensus W-determinant (Table 1; IgM41, bold). The W-peptides formed a few homology groups, which are shown outlined (Table 1; IgM41). Three W peptides, T7-LWNS, T7-PEWQ and T7-SWT, were truncated by random stop-codons. Three peptides contained noW residues (Table 1E; IgM41).

Thirteen of 20 peptides selected with IgM58 contained the dipeptide TP (TP-determinant), which was located one or two residues away from the C-terminus. The TP-peptides also formed a few homology groups (Table 1; IgM58). Two peptides from this selection contained only one consensus residue (Table 1D; IgM58). The remaining five peptides were devoid of consensus residues (Table 1E; IgM58).

Seventeen of 20 peptides selected with IgM60 contained an L residue in the −4 position (L-determinant). Eight of these peptides had a G residue and five peptides, either N or Q residue in the −5 position (Table 1, IgM60). Two peptides from this selection were devoid of L-determinants and one peptide was truncated by a stop-codon (Table 1D; IgM60).

TABLE 1
Peptides selected for binding to IgM104 and polyspecific IgM in TBS-BT

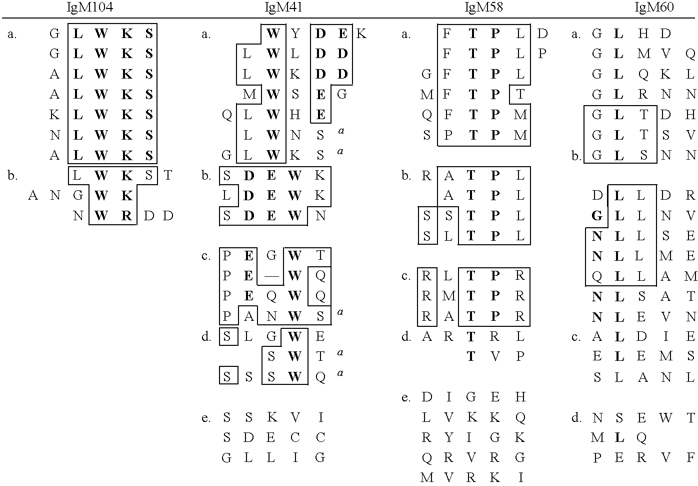

Consensus resides are shown in bold.
Homologous sequences are outlined.
^{a}W-containing peptides without D/E resides.

Polyspecific IgM Abs in solution predominantly bind to cognate selected peptides carrying consensus determinants. The binding specificity of free IgM was significantly higher than that of immobilized IgM. Thus, only five of 20 peptides selected with immobilized IgM41 strongly bound free IgM41 in ELISA. Notably, all five peptides contained W-determinants (Table 2; IgM41, “+”). Only 15 of 20 peptides selected with immobilized IgM58 bound free IgM58, with 13 reactive peptides containing the consensus TP-determinant (Table 2; IgM58, “+”). Unlike IgM41, IgM58 reacted with two peptides devoid of the consensus determinant (Table 2; IgM58, outlined). IgM60 bound 15 of 19 cognate peptides, with 14 peptides containing the L-determinant (Table 2, IgM60). Similarly to IgM58, IgM60 reacted with a peptide devoid of the consensus determinant (Table 2; IgM60, outlined). The binding avidity shown by all three Abs in reactions with cognate peptides was comparable to their avidity in reactions with screening Ags (FIGS. 1A-D, dashed lines with symbols).

TABLE 2
Binding specificity of polyspecific IgM in solution
to cognate selected peptides

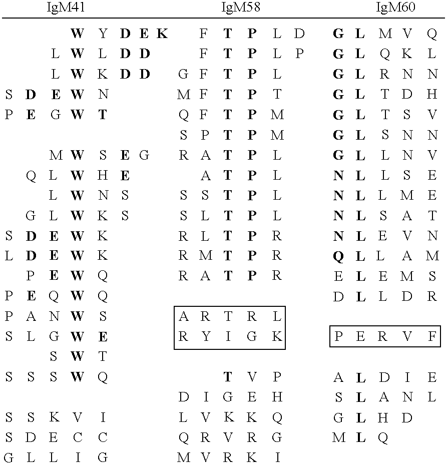

Consensus resides are shown in bold.
IgM-binding peptides devoid of consensus determinants are outlined.

The IgM complexes with peptides carrying consensus determinants invariably activated complement (FIG. 1D). Hence, these complexes formed in a physiologically meaningful manner (Perkins et al., 1991; Thornton et al., 1994). This was consistent with the inventors' observations that complement inactivated a T7 phage display library in serum and blood in an IgM-dependent and peptide-specific fashion (Sokoloff et al., 2001; 2000).

W-determinants are universally recognized by natural IgM antibodies. Additional selection experiments were conducted under the same conditions using six more monoclonal IgM antibodies derived from naïve Balb/c mice. One of these antibodies, IgM42, was sequenced and found to be encoded by germline genes. It was found that all antibodies tested reacted predominantly with W determinants. The W determinants identified in these experiments belonged to the same homology groups that were observed for IgM41 described above. These selections results suggested that W determinants are universal antigenic determinananats recognized by natural IgM antibodies. This conclusion was corroborated by experiments with polyclonal IgM present in normal serum, which is thought to be composed mostly of natural antibodies. Immobilized phage displaying W determinants efficiently bound normal serum IgM from mouse and human serum. With mouse Balb/c serum, the efficiency of IgM binding was approximately the same as with immobilized anti-IgM antibody (mu chain-specific). Further evidence in support of the universal nature of W determinants was obtained by conducting selection experiments with polyclonal serum IgM immobilized onto ELISA plates from normal serum. W determinants vastly dominated in the population of selected peptides that reacted with serum IgM. The same W determinant homology groups as observed in the selections with monoclonal IgM antibodies were identified.

W determinants are mimicked by IPA and structurally related compounds. The common structural motif observed in W determinants is the presence of Trp and one or two negatively charged amino acid residues. In this regard, W determinants are mimicked by auxins, which are naturally occurring plant growth hormones that include IPA, indole-3-butiric acid and indole-3-acetic acid. Testing IPA was particularly interesting since it is non-toxic and has been suggested for human trials as an antioxidant. The addition of IPA into the incubation medium significantly inhibited the binding of natural IgM to W determinants. The inhibition of IgM binding to W determinants was also observed with indole-3-acetic acid, indole-3-butiric acid and indole-3-acrylic acid, indicating that a spacer of variable length and rigidity can be used to separate the indole and negatively charged groups in simple chemical mimetics of W determinants. The inhibition of IgM binding to W determinants was also observed with N-(indolylacetyl)-L-Ala, N-(indolylacetyl)-DL-Asp and N-(indolylacetyl)-L-Ile. In this compounds, an amino acid residue was a donor of a free carnoxyl group. 2-Benzimidazolepropionic acid also inhibited IgM binding to W determinants to a significant degree, indicating that structural analogues of the indole group are also effective and that the modification position of the indole group, used to introduce a negatively-charged group may vary.

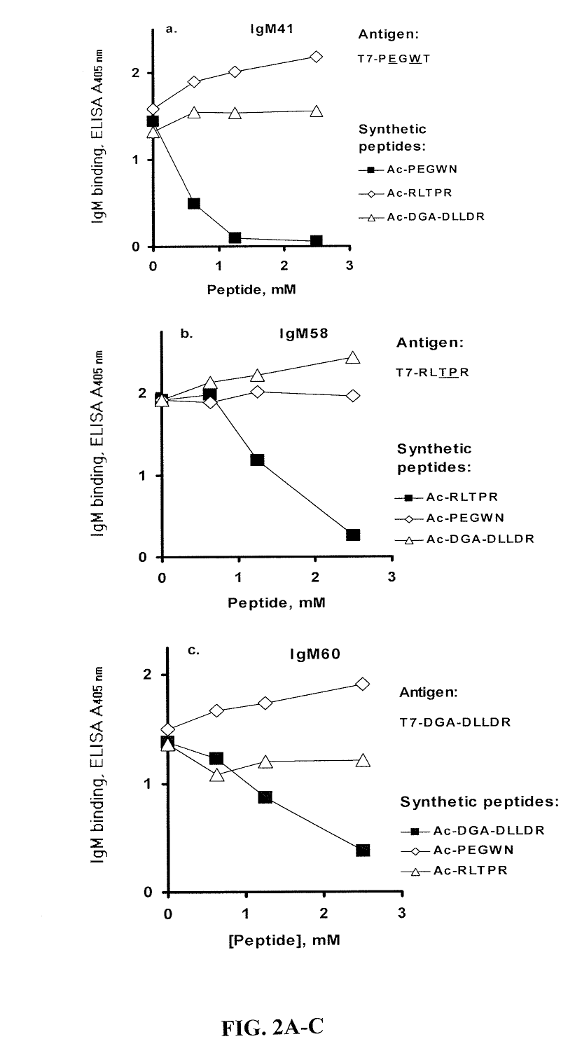

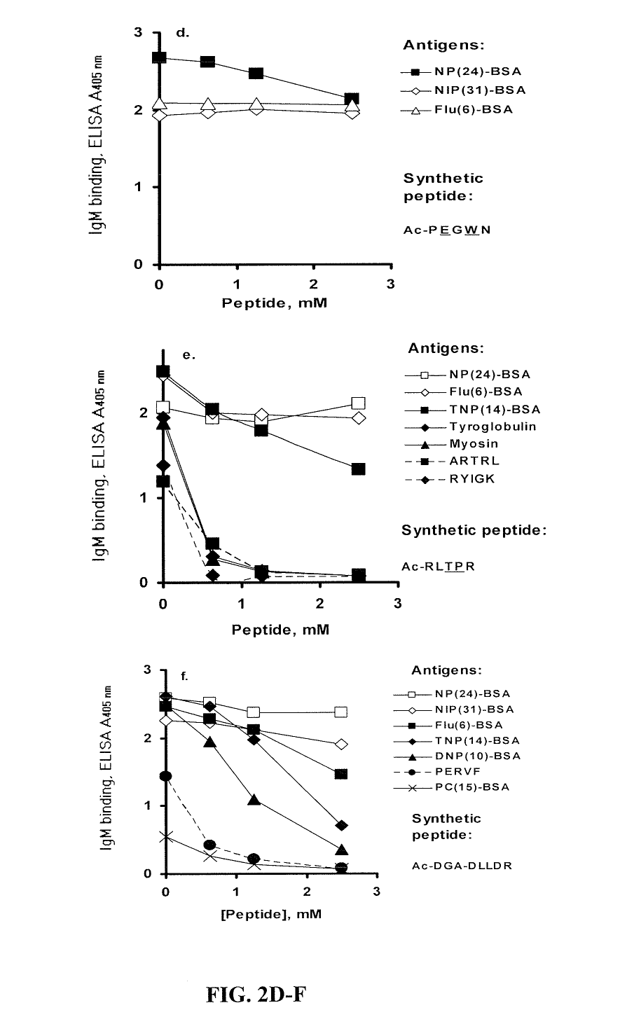

Cognate selected peptides compete with common screening Ags for IgM binding. The relationship between the IgM sites binding selected peptides and those binding screening Ags was analyzed in competition experiments with free synthetic peptides that were used as a substitute for phage-displayed peptides to minimize steric hindrance effects. The utility of synthetic peptides for this purpose was confirmed by examining their competition with phage-displayed peptides for binding cognate IgM. IgM41 binding to the peptide T7-PEGWT was completely inhibited by the homologous synthetic peptide Ac-PEGWN (FIG. 2A, closed symbols). No inhibition of IgM41 binding was observed when the same displayed peptide was paired with the unrelated synthetic peptides Ac-RLTPR and Ac-DGA-DLLDR that reacted with IgM58 and IgM60, respectively (FIG. 2A, open symbols). Likewise, IgM58 binding to the cognate peptide T7-RLTPR was inhibited by the homologous synthetic peptide Ac-RLTPR but not by the unrelated peptides Ac-PEGWN and Ac-DGA-DLLDR (FIG. 2B). The synthetic peptide Ac-DLLDR just weakly inhibited IgM60 binding to the peptide T7-DLLDR. Additional selection experiments showed that the L-determinant was recognized more efficiently when it was flanked by the spacer sequence DGA (data not shown). The new synthetic peptide Ac-DGA-DLLDR significantly inhibited IgM60 binding to the peptide T7-DLLDR (FIG. 2C, closed symbols), while the unrelated peptides Ac-PEGWN and Ac-RLTPR were ineffective (FIG. 2C, open symbols).

The peptide Ac-PEGWN just weakly inhibited IgM41 binding to NP(24)-BSA and had no effect on its binding to NIP(31)-BSA or Flu(6)-BSA (FIG. 2C). In contrast, the peptide Ac-RLTPR completely inhibited IgM58 binding to thyroglobulin and myosin and moderately, to TNP(14)-BSA (FIG. 2E, closed symbols). This peptide also completely inhibited IgM58 binding to the cognate peptides T7-RYIGK and T7-ARTRL, which were devoid of the consensus TP determinant (FIG. 2E, dashed lines). Similarly, the peptide Ac-DGA-DLLDR significantly reduced IgM60 binding to TNP(14)-BSA, DNP(10)-BSA and PC(15)-BSA (FIG. 2F, closed symbols). This peptide also completely inhibited IgM60 binding to the peptide PERVF, which was devoid of the consensus L-determinant (FIG. 2F, dashed line). The IgM-binding competition observed between synthetic peptides and screening Ags suggested that these two types of Ags reacted with the same or overlapping IgM sites. However, it could not be ruled out that the synthetic peptides bound to distinct sites and “locked” IgM molecules in conformational states that did not support polyspecificity (Foote and Milstein, 1994; James et al., 2003).

Undiluted serum does not inhibit IgM binding to cognate selected peptides containing consensus determinants. The reactivity of polyspecific IgM Abs with cognate selected peptides in serum was first examined by conducting selection experiments in 90% FBS^{HI}. IgM from culture supernatants was used in these selections to prevent IgM-serum interactions that could be caused by IgM purification. The peptides selected in 90% FBS^{HI} contained exactly the same consensus determinants as those selected in TBS-BT (Table 3; compare with Table 1). Thus, the IgM reactivity with peptides carrying consensus determinants was evidently medium-independent and was not associated with the use of a particular IgM isolation procedure.

TABLE 3
Peptides selected for binding to polyspecific IgM in 90% FBS^{HI}

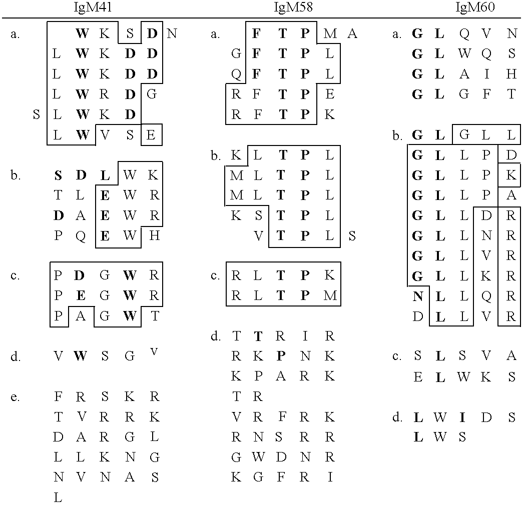

Consensus resides are shown in bold.
Homologous sequences are outlined..

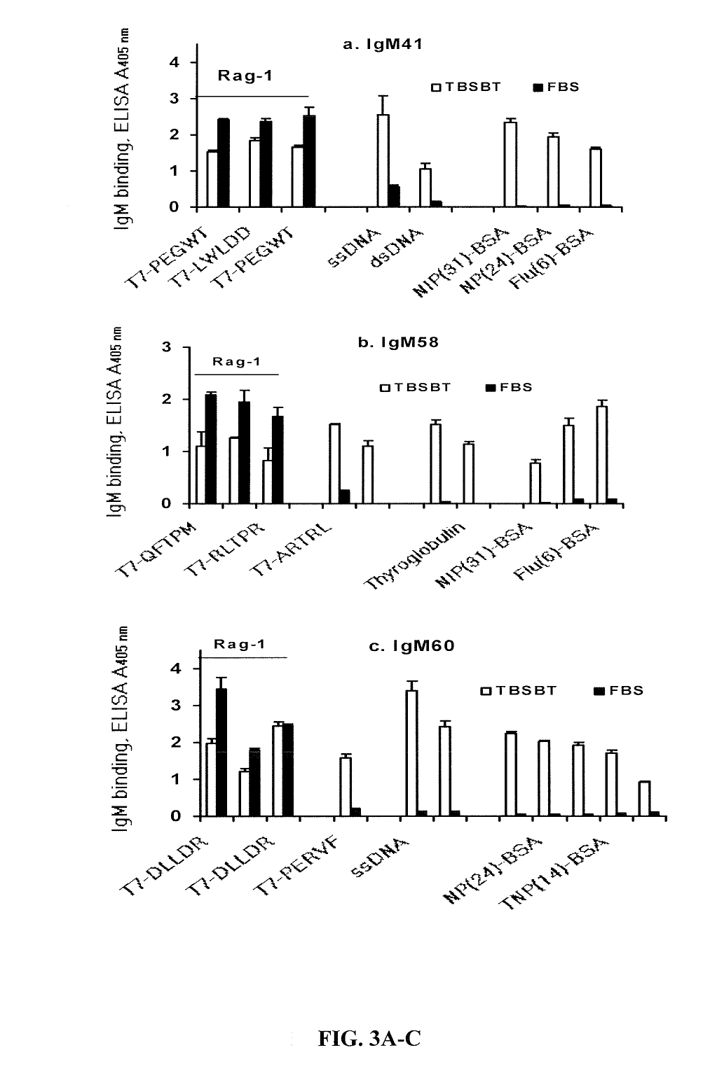

In keeping with the selection results, IgM binding to peptides displaying consensus determinants was unaffected or even slightly stimulated by 95-97% FBS_{EDTA} and FBS^{HI} (FIGS. 3A-C, closed bars). This suggested that the IgM Abs did not significantly react with FBS macromolecules despite their polyspecificity in regard to screening Ags. The possibility that the Abs retained their full binding capacity with respect to phage displayed peptides while reacting with FBS macromolecules seemed unlikely. In the experiments modeling IgM-FBS interactions, the addition of NP(24)-BSA, NIP(31)-BSA or Flu(6)-BSA(1 mg/ml) to TBS-BT reduced the efficiency of IgM41 binding to the cognate peptide T7-PEGWT by 50-75%. IgM60 binding to the cognate peptide T7-DLLDR was similarly inhibited by Flu(6)-BSA and TNP(14)-BSA. NP(24)-BSA and NIP(31)-BSA completely inhibited IgM60 binding at concentrations below 0.1 mg/ml. The efficiency of IgM58 binding to the cognate peptide T7-QFTPM was inhibited by 30-40% by TNP(14)-BSA. Finally, the binding of all three Abs was abolished by Fab Abs (˜110 μg/ml) directed against the mouse Fab region (data not shown).

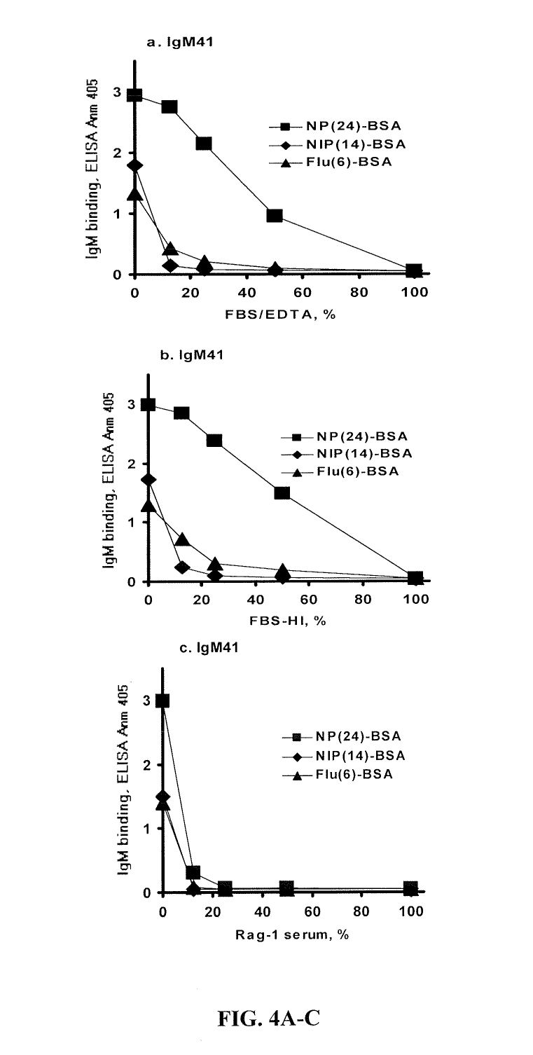

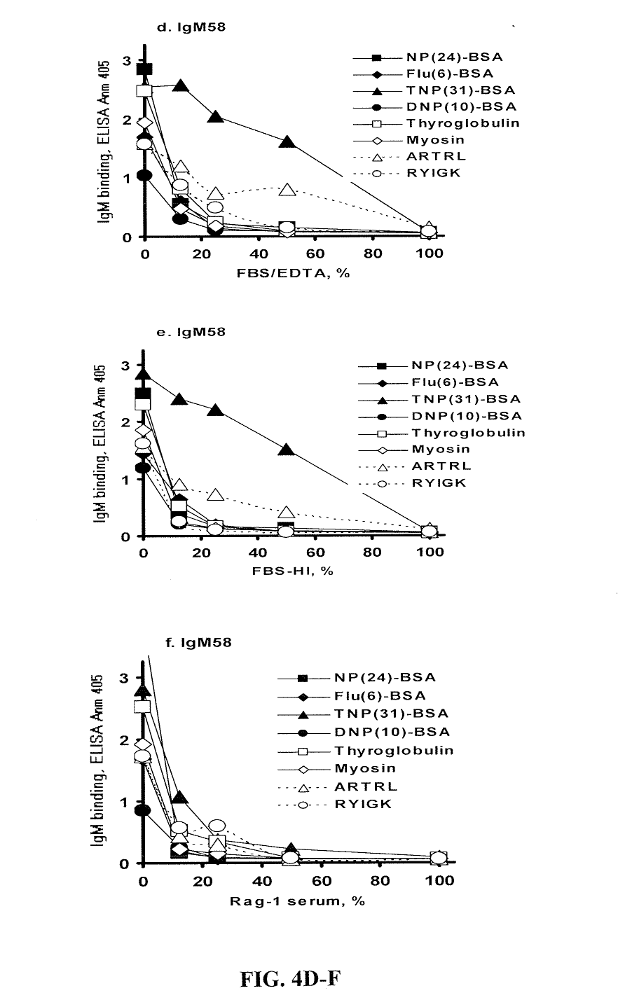

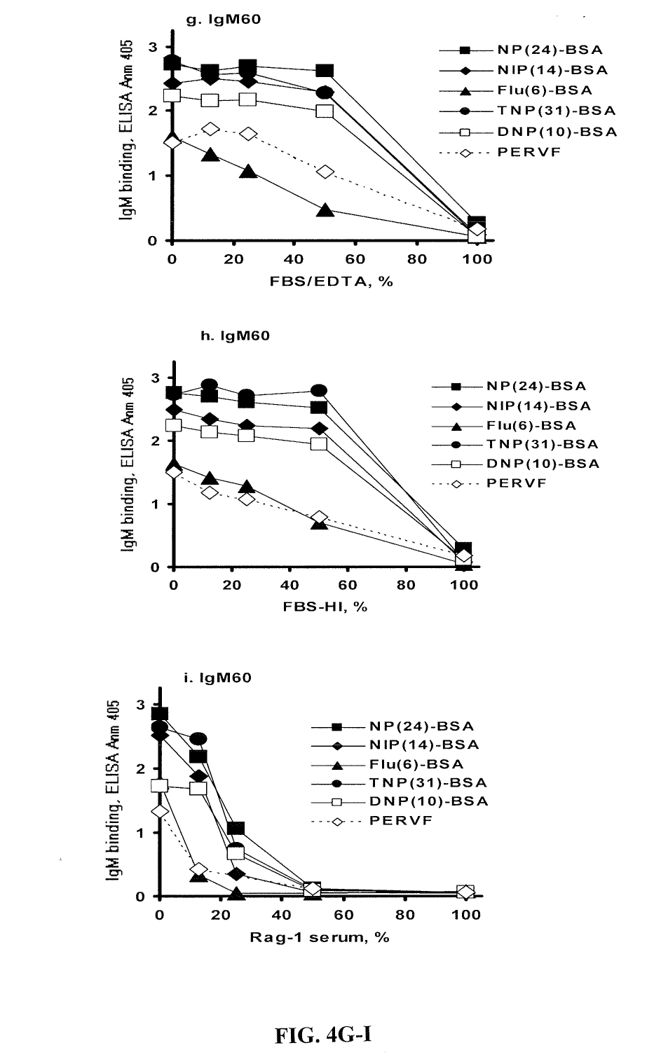

Undiluted serum inhibits IgM binding to cognate selected peptides devoid of consensus determinants. IgM58 binding to the cognate peptides T7-ARTRL and T7-RYIGK, both of which were lacking the consensus TP-determinant, was strongly inhibited by FBS_{EDTA} (FIG. 3B, asterisk-labeled bars). FBS^{EDTA} similarly suppressed IgM60 binding to the peptide T7-PERVF, which was devoid of the consensus L-determinant (FIG. 3C, asterisk-labeled bar). FBS^{EDTA} and FBS^{HI} inhibited IgM binding to these peptides with similar efficiency (FIGS. 4A-I, dashed lines). Thus, neither FBS heat inactivation nor chelation of FBS metal cations by EDTA contributed to the inhibition process.

No IgM binding inhibition was observed when the peptides whose IgM-binding activity was inhibited by FBS were preincubated with 100% FBS^{HI} for 30 min and then washed with TBS and allowed to react with IgM in TBS-BT. This ruled out detachment or irreversible blocking of immobilized phage as a cause of IgM-binding inhibition (data not shown). The IgM-binding inhibition also could be trivially explained by the blocking of peptides by low-level natural Abs present in FBS. However, IgM binding was inhibited even stronger by immunoglobulin-free Rag-1 mouse serum (FIGS. 4A-I). Like FBS, Rag-1 serum did not inhibit IgM binding to cognate peptides that carried consensus determinants (FIGS. 3A-C, “Rag-1”-labeled bars).

The inhibition of IgM binding to peptides devoid of consensus determinants could be due to differential blocking of these peptides by serum macromolecules or due to IgM conformational changes induced by the serum environment. In the former case, the peptides RYIGK and PERVF could represent examples of broadly reactive epitopes. Both of these peptides featured distinctive sequences, RYI and RVF, which contained one positively charged and two hydrophobic residues, one of which had an aromatic side chain. No similar sequences were found among 24 selected peptides that did not react with free IgM (Table 2).

Undiluted serum inhibits IgM binding to conventional screening Ags. FBS^{HI}, FBS_{EDTA} and Rag-1 serum invariably inhibited IgM binding to screening Ags (FIGS. 3A-C and 4A-I). An insignificant decrease in IgM binding was observed when screening Ags were preincubated for 30 min with 100% FBS^{HI} and then washed with TBS and incubated with IgM in TBS-BT. This ruled out detachment or irreversible blocking of screening Ags in the presence of serum (data not shown).

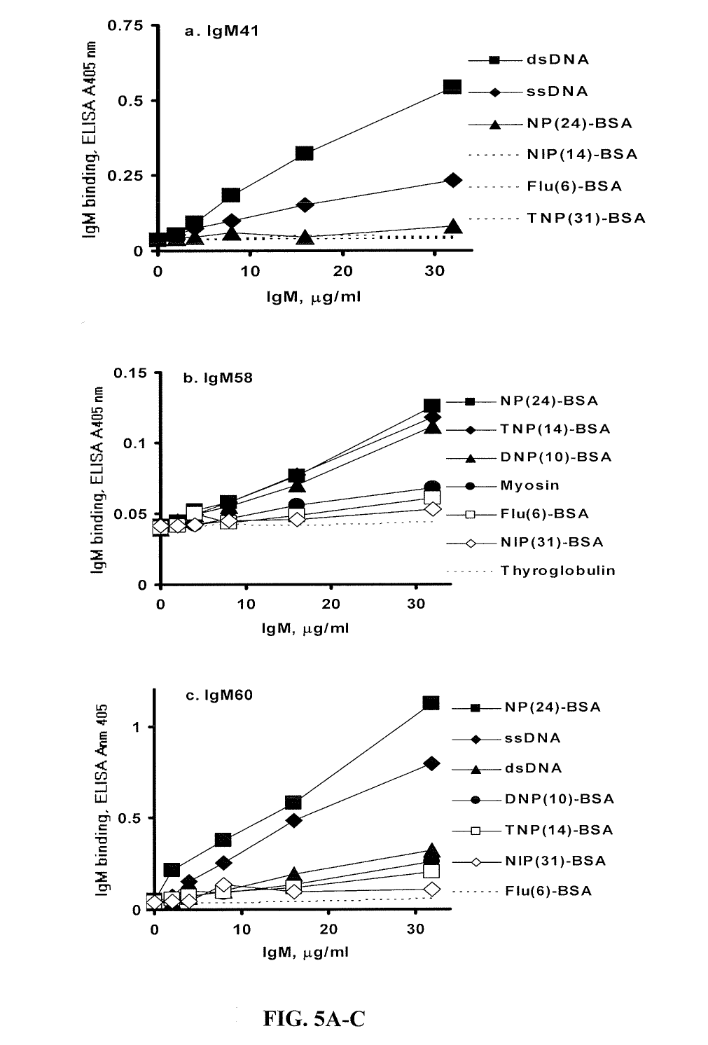

Increasing the concentration of IgM reduced the efficiency of the IgM-binding inhibition by FBS in an Ag-dependent manner. There was a noticeable increase in the amount of IgM41 bound to ssDNA and dsDNA, but not to other Ags (FIG. 5A). IgM58 showed no significant binding at any concentration (FIG. 5B). An increase in the amount of bound IgM60 was observed with NP(24)-BSA and ssDNA (FIG. 5C). Even at the highest IgM concentration, 32 μg/ml, the binding activity of all three IgM Abs in FBS was by far lower than that in TBS-BT (compare FIGS. 1A-D and 5A-C).

The inhibition of IgM binding to screening Ags could be attributed to blocking of these Ags by serum macromolecules. Alternatively, IgM could undergo serum-induced conformational changes that restrict polyspecificity. As judged from the shape of binding isotherms, the conspicuous absence of IgM-binding inhibition observed with the peptides carrying consensus determinants was not due to their higher binding avidity (FIGS. 1A-D, dashed lines with symbols).

A large portion of both non-stimulated and LPS-stimulated B cells react with W-determinats in ELISPOT. Up to 38% of non-stimulated cells secreting IgM reacted with phage-displayed W determinants, while only 0.35% of these cells reacted with control phage that displayed negatively charged peptides lacking Trp residues. The presence of 2.5 mM IPA in the medium reduced the number cells binding to W determinants by 70-80%. The number of B cells reacting with W determinants increased by over 20-fold as a result of LPS stimulation, while the number of cells reacting with the control peptide was essentially unaffected. As many as 70-80% of LPS-stimulated cells reacted with W-determinants. These experiments show that the majority of B cells in the spleen and peritoneal cavity express antigen receptors (surface immunoglobulins) that react with W determinants, which further supports view that W determinants are universal antigenic determinants recognized by otherwise unrelated immunoglobulins.

IPA prevents the development of autoimmunity in MLR/1pr mice. The main manifestation of autoimmunity in MRL/1pr mice is a dramatic increase in the size of their lymph nodes (up to 102-fold) and spleens (several-fold). All of these expected changes were observed in mice that were kept on normal water. In contrast, very little lymph node and spleen enlargement, if any, was observed in mice that received 20 mM IPA instead of water. The involvement of B cells in the development of autoimmunity in MRL/1pr mice is a well-known phenomenon. It appears that IPA blocks the antigenic stimulation of naïve B cells that is required for their progression towards an autoimmune state.

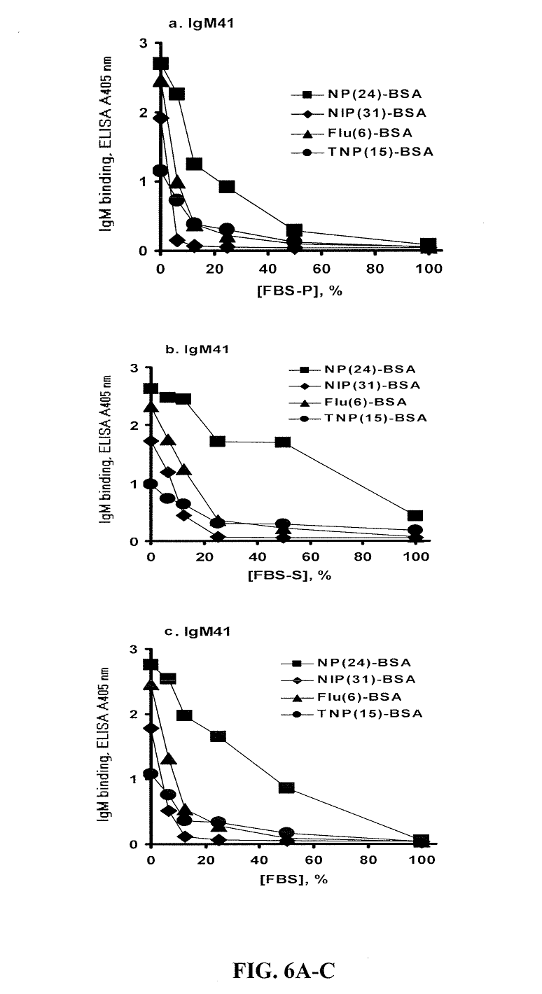

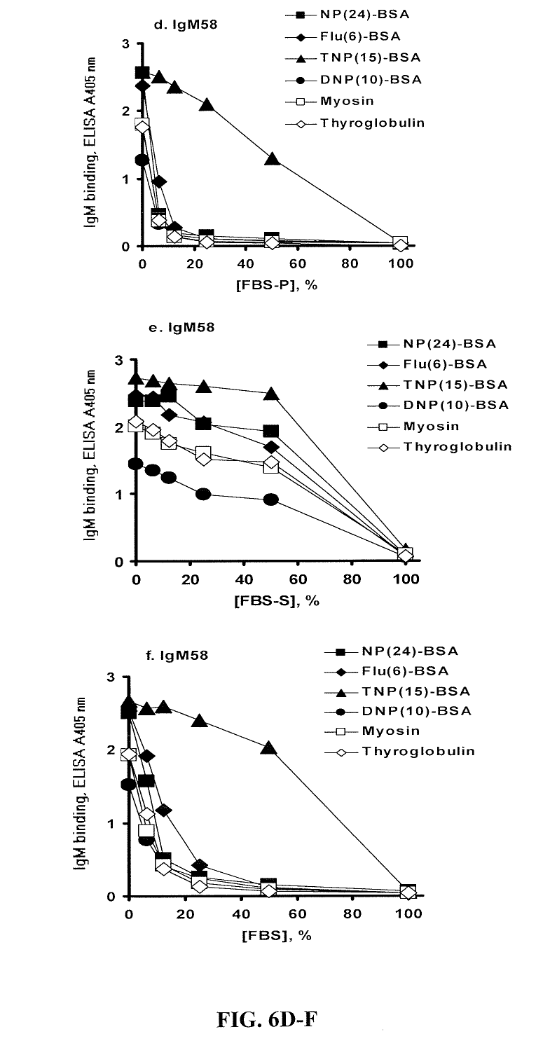

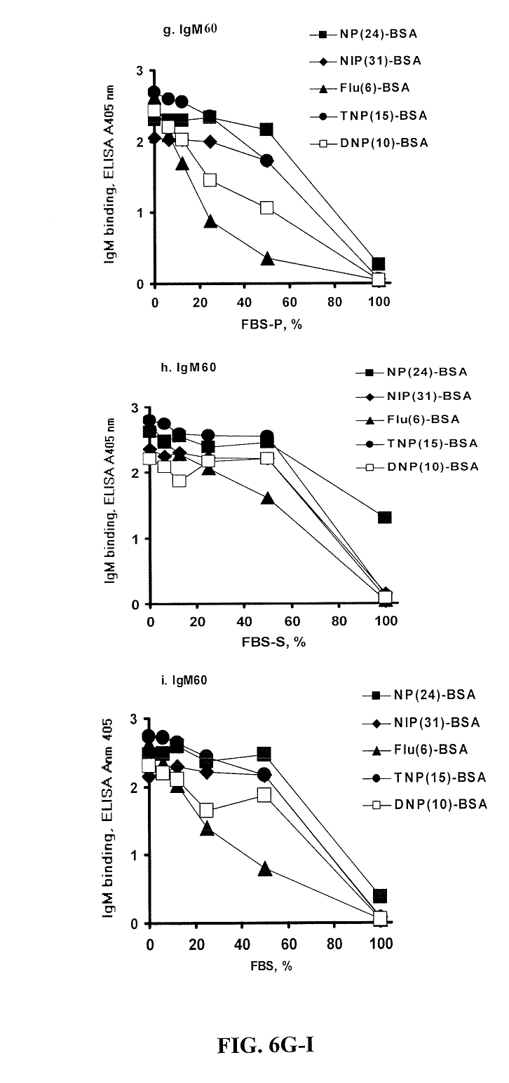

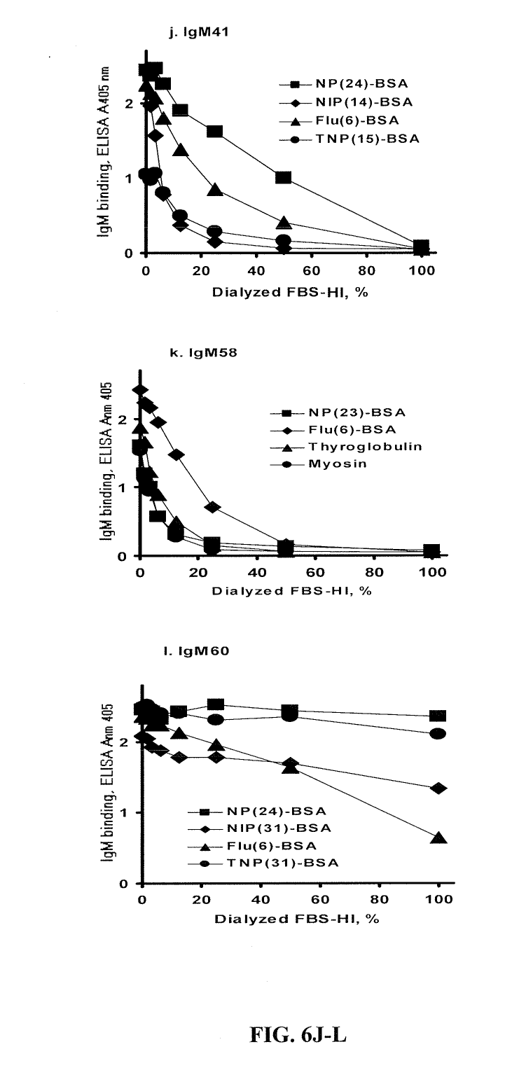

Different serum factors contribute to the loss of IgM polyspecificity. The FBS^{HI} fraction precipitated by 45% ammonium sulfate (FBS-P) inhibited IgM41 binding to screening Ags nearly as effectively as non-fractionated FBS^{HI} (FIGS. 6A and 6C). The FBS supernatant fraction (FBS-S) was approximately two-fold less inhibitory (FIG. 6B). IgM60 binding to screening Ags was inhibited by FBS-P and FBS-S with approximately the same efficiency as observed for IgM41 (FIGS. 6G-I). IgM58 binding to all tested Ags, except TNP(15)-BSA, was inhibited by FBS-P approximately two- and 5-10-fold more effectively than by FBS^{HI} and FBS-S, respectively (FIGS. 6D-F). Thus, the FBS inhibitory activity seemed to be at least partly associated with serum macromolecules.

Involvement of low-molecular-weight FBS components in inhibiting IgM-binding was suggested by the experiments with extensively dialyzed FBS. This FBS inhibited IgM41 binding to screening Ags somewhat less effectively than the moderately dialyzed FBS described above (FIGS. 6J and 6C, respectively). IgM58-binding was inhibited by these sera with approximately the same efficiency (FIGS. 6K and 6F, respectively). However, the extensively dialyzed FBS did not inhibit IgM60 binding to NP(24)-BSA or TNP(15)-BSA and only weakly inhibited its binding to NP(31)-BSA and Flu(6)-BSA (FIGS. 6L and 6I). This suggested that the blocking of IgM60 polyspecificity required the presence of low-molecular-weight FBS components that could be removed by extensive dialysis. Alternatively, IgM60 could be more sensitive than IgM41 and IgM60 to changes in the redox state of FBS macromolecules, which might occur during extensive dialysis (McIntyre, 2004).

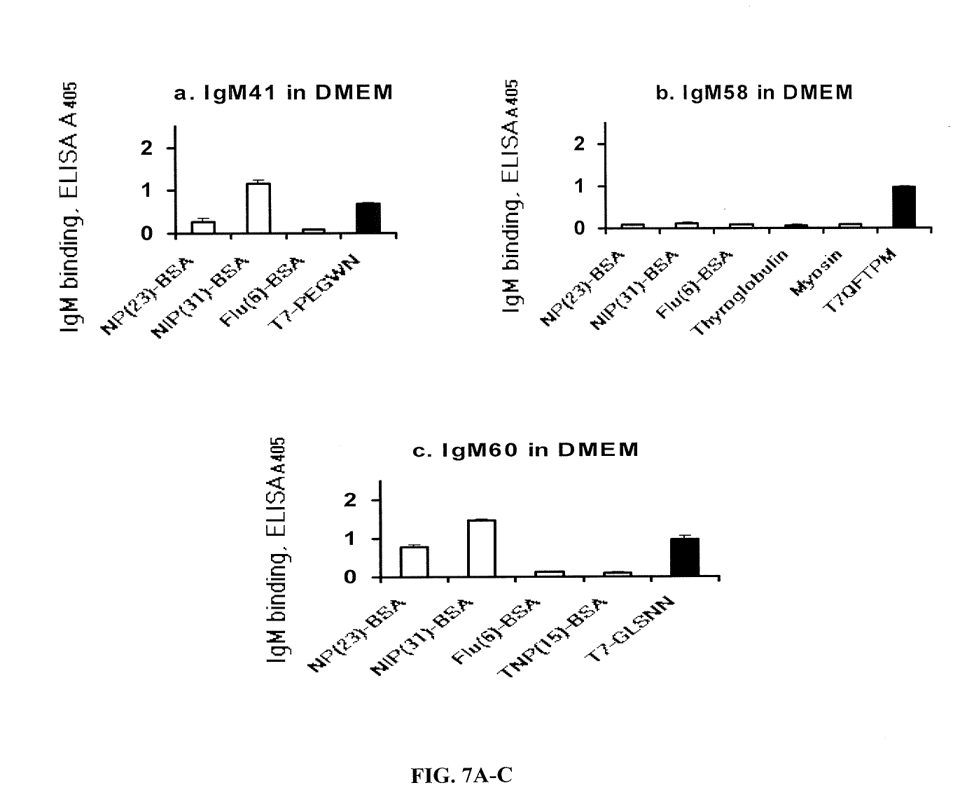

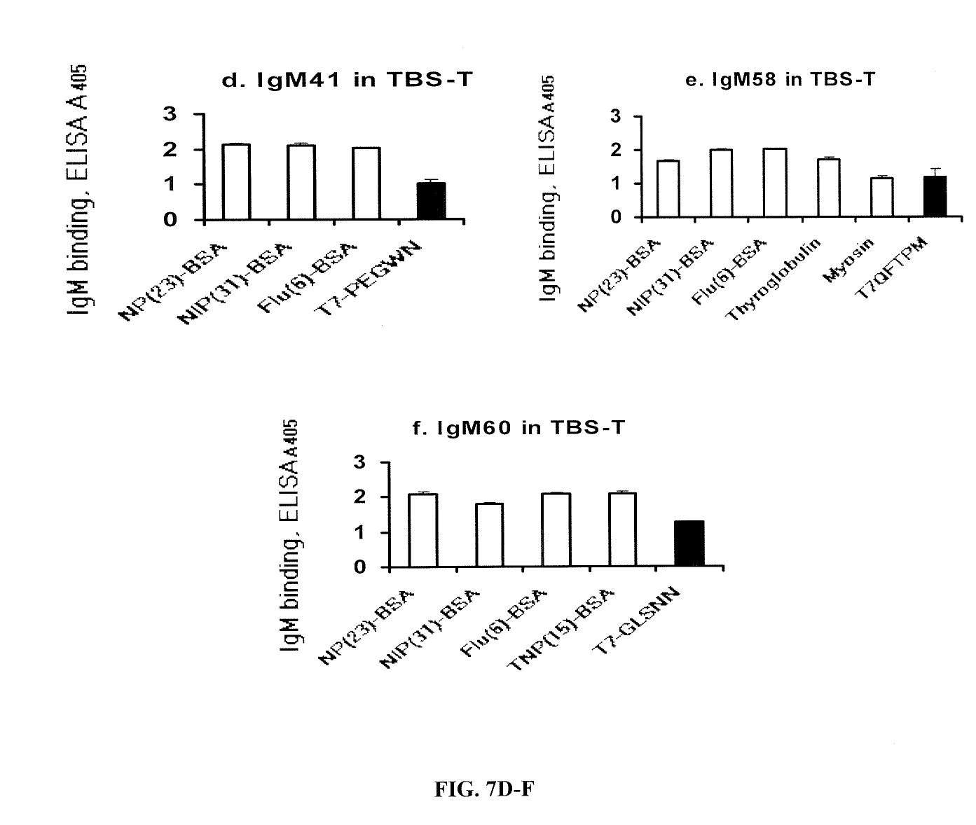

The contribution of low-molecular-weight serum components to the loss of IgM polyspecificity was also suggested by the results of IgM-binding experiments conducted with DMEM as a binding medium. TBS/0.05% Tween-20 (TBS-T), which supported polyspecific IgM binding as efficiently as TBS-BT (data not shown), was used as a DMEM counterpart. The use of DMEM significantly reduced the expression of polyspecificity by all three Abs (FIGS. 7A-F).

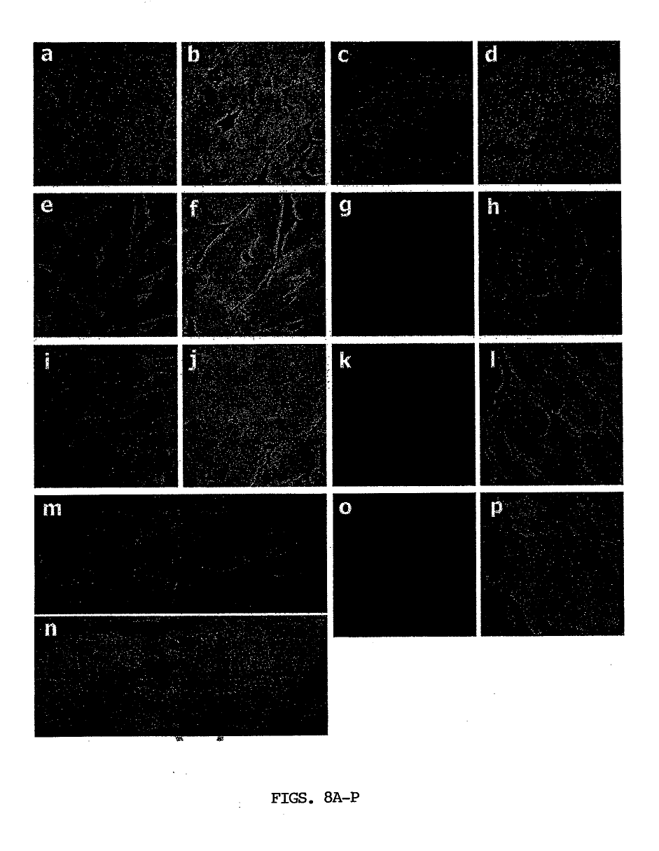

Undiluted serum blocks natural IgM polyspecificity in reactions with muscle tissue Ags. To address the concern that the loss of IgM polyspecificity in serum was a specific phenomenon, restricted to certain displayed peptides and highly purified screening Ags, the inventors conducted IgM-binding experiments using mouse muscle tissue as a source of naturally occurring endogenous Ags (Zhang et al., 2006). All three Abs produced extensive staining when incubated with unfixed skeletal muscle sections in TBS-B, with Tween-20 omitted to avoid Ag extraction (FIGS. 8A, 8E and 8I). The use of undiluted FBS^{HI} instead of TBS-B led to dramatic changes in IgM reactivity. The staining produced by IgM58 and IgM60 nearly completely disappeared (FIGS. 8G and 8K, respectively). The weak residual signal near the sarcolemma was virtually indistinguishable from the background staining attributed to endogenous IgM (FIG. 80). The effect of FBS^{HI} on IgM41 was more interesting. While the bulk of the IgM41 staining disappeared, the remaining staining was apparently specific for myofilaments (FIGS. 8C and 8M). In the individual optical sections (0.4 μm), the IgM staining pattern (red) was continuous with the staining pattern of Phalloidin that bound to actin (green; FIG. 8N). The general myofiber morphology, assessed by staining sections for actin (green) and nuclei (blue) in addition to IgM, was similarly preserved in all samples (FIGS. 8A-N).

These experiments with tissue sections showed that the loss of IgM polyspecificity in serum was a general phenomenon, observed with both highly purified and naturally occurring Ags. This conclusion was supported by our preliminary data obtained from testing seven more natural IgM Abs derived from Balb/c mice. Five new Abs stained muscle sections in TBS-B, with three of them losing reactivity and two others showing narrow specificity in FBS (data not shown).

Additional peptides define other consensus sequences. Table 4 shows additional peptides from mouse and human selections using a similar selection system as compared to that described above. Some were selected using phage grown in E. coli BLT5616 cells that express an inducible plasmid producing the wild-type phage coat protein 10A that does not contain reactive sequences. As a result, hybrid phage particles are formed that have a coat composed of both protein 10B (˜10%), which displays a peptide, and protein 10A (˜90%), whose presence decreases the density of displayed peptides. This reduces weak non-specific interactions that are more dependent on avidity (the stability of the complex regardless of the nature of bonds that keep it together) than on affinity (the strength of ligand-receptor binding). This approach avoids selecting clones containing highly positively-charged peptides, but without sequence specificity.

Table 4 shows peptides with the following structural features. First, column A shows a substantial number of peptides that have a W and either F or Y or H residue. All of these peptides contain one or two D/E residues. The W and F/H/Y are almost always next to each other. One exception is marked with an asterisk. The majority of these peptides have a D/E residue next to the W—F/H/Y cluster. They can be viewed as the covered W-peptides containing an amino acid residue with an aromatic side chain (H/Y/F). These peptides occur in both mouse and human selections. Column B shows peptides found mostly in human selections. These contain two Y/F/H residues and one D/E residue in all possible combinations. Column C shows peptides similar to those in column B, but with just one F/H/Y residue. Again, the F/H/Y residue and the D/E residue(s) produce various positional combinations.

TABLE 4
Selected peptides reacting with monoclonal and
polyclonal Nabs antibodies

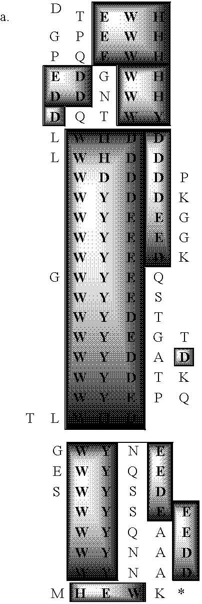

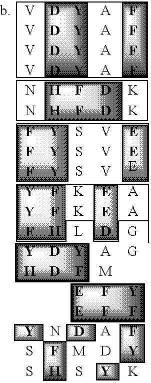

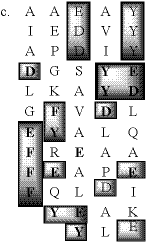

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,440,013
U.S. Pat. No. 5,446,128
U.S. Pat. No. 5,475,085
U.S. Pat. No. 5,618,914
U.S. Pat. No. 5,670,155
U.S. Pat. No. 5,672,681
U.S. Pat. No. 5,674,976
U.S. Pat. No. 5,710,245
U.S. Pat. No. 5,840,833
U.S. Pat. No. 5,859,184
U.S. Pat. No. 5,929,237
Arend and Dayer, Arthritis Rheum., 38:151-160, 1995.
Arend et al., Annu. Rev. Immunol., 16:27-55, 1998.
Armstrong and Quigley, Dev. Comp. Immunol., 23:375-390, 1999.
Baichwal and Sugden, In: Gene Transfer, Kucherlapati (Ed.), NY, Plenum Press, 117-148, 1986.
Barany and Merrifield, In: The Peptides, Gross and Meienhofer (Eds.), 1-284, Academic Press, NY, 1979.
Baumgarth et al., J. Exp. Med., 192:271-280, 2000.
Boes, Mol. Immunol., 37, 1141-1149, 2000.
Bouvet et al., J. Autoimmun., 16:163-172, 2001.
Burger and Dayer, Neurology, 45(6S-6):S39-43, 1995.
Capaldi et al., Biochem. Biophys. Res. Comm., 74(2):425-433, 1977.
Casali and Schettino, Curr. Top. Microbiol. Immunol., 210:167-179, 1996.
Chen and Okayama, Mol. Cell. Biol., 7(8):2745-2752, 1987.
Chen et al., J. Immunol., 147:2359-2367, 1991.
Coupar et al., Gene, 68:1-10, 1988.
Diaw et al., J. Immunol., 158:968-976, 1997.
Dighiero et al., J. Immunol., 131:2267-2272, 1983.
Dimitrov et al., J. Biol. Chem., 281:439-446, 2006.
Dinarello, Int. Rev. Immunol., 16:457-499, 1998.
Eastgate et al., Lancet, 2:706-709, 1988.
Ehrenstein et al., J. Exp. Med., 191:1253-1258, 2000.
Ehrenstein et al., Proc. Natl. Acad. Sci. USA, 95:10089-10093, 1998.
Ellis et al., Clin. Exp. Immunol., 33:136-141, 1978.
Ellis, Trends Biochem. Sci., 26:597-604, 2001.
Fechheimer, et al., Proc Natl. Acad. Sci. USA, 84:8463-8467, 1987.
Firestein et al., Arthritis Rheum., 37:644-652, 1994.
Foote and Milstein, Proc. Natl. Acad. Sci. USA, 91:10370-10374, 1994.
Fraley et al., Proc. Natl. Acad. Sci. USA, 76:3348-3352, 1979.
Fujikawa et al., Ann. Rheum. Dis., 54:318-320, 1995.
Gopal, Mol. Cell. Biol., 5:1188-1190, 1985.
Graham and Van Der Eb, Virology, 52:456-467, 1973.
Hannum et al., Nature, 343:336-340, 1990.
Harland and Weintraub, J. Cell Biol., 101(3):1094-1099, 1985.
Haspel et al., Nature, 304:73-76, 1983.
James et al., Science, 299:1362-1367, 2003.
Johannesson et al., J. Med. Chem., 42(22):4524-4537, 1999.
Johnson et al., In: Biotechnology And Pharmacy, Pezzuto et al. (Eds.), Chapman and Hall, NY, 1993.
Jonsson et al., Br. J. Rheumatol., 32(7):578-581 1993.
Jonsson et al., Oral Dis., 8(3):130-140, 2002.
Jonsson et al., Trends Immunol., 22(12):653-654, 2001.
Kahle et al., Ann. Rheum. Dis., 51:731-734, 1992.
Kuehn, JAMA, 294(23):2960, 2005.
Lipsky, In: Harrison's principles of internal medicine, Fauci et al. (Eds.), 14^{th} Ed., NY, McGraw-Hill, 1880-1888, 1998.
Manivel et al., J. Immunol., 169:888-897, 2002.
McIntyre, Thromb. Res., 114:579-587, 2004.
McMahon and O'Kennedy, J. Immunol. Methods, 241:1-10, 2000.
Merrifield, Science, 232(4748):341-347, 1986.
Nevens et al., J. Chromatogr. A, 597:247-256, 1992.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Notkins, Trends Immunol., 25:174-179, 2004.
Ochsenbein et al., Science, 286:2156-2159, 1999.
Owen and Campbell, J. Immunol., 160:1436-1443, 1998.
PCT Appln. WO 84/03564
Perkins et al., J. Mol. Biol., 221:1345-1366, 1991.
Potter et al., Proc. Natl. Acad. Sci. USA, 81:7161-7165, 1984.
Prieur et al., Lancet., 2:1240-1242, 1987.
Remington's Pharmaceutical Sciences, 18th Ed. Mack Printing Company, 1990.
Ridgeway, In: Vectors: A survey of molecular cloning vectors and their uses, Rodriguez and Denhardt (Eds.), Stoneham:Butterworth, 467-492, 1988.
Rippe et al., Mol. Cell. Biol., 10:689-695, 1990.
Rooney et al., Rheumatol. Int., 10:217-219, 1990.
Salomonsson and Jonsson, Arthritis Rheum., 48(11):3187-3201, 2003.
Salomonsson et al., Scand. J. Immunol., 55(4):336-342, 2002.
Schepers et al., Biochemistry, 17:2239-2245, 1978.
Schwab and Bosshard, J. Immunol. Methods, 147:125-134, 1992.
Sebestyen et al., J Gene Med., 8:852-873, 2006.
Seidl et al., Int. Immunol., 9:689-702, 1997.
Shan et al., J. Immunol., 153:5104-5120, 1994.
Sokoloff et al., Immunology, 112:237-249, 2004.
Sokoloff et al., Mol. Ther., 2:131-139, 2000.
Sokoloff et al., Mol. Ther., 3:821-830, 2001.
Stewart and Young, In: Solid Phase Peptide Synthesis, 2d. ed., Pierce Chemical Co., 1984.
Tam et al., J. Am. Chem. Soc., 105:6442, 1983.
Temin, In: Gene Transfer, Kucherlapati (Ed.), NY, Plenum Press, 149-188, 1986.
Thornton et al., J. Immunol., 152:1727-1737, 1994.
Tissot et al., Electrophoresis, 23:1203-1206, 2002.
Tur-Kaspa et al., Mol. Cell. Biol., 6:716-718, 1986.
van den Berg, Semin. Arthritis Rheum., 30(5S-2):7-16, 2001.
Vita et al., Biopolymers, 47:93-100, 1998.
Weisshoff et al., Eur. J. Biochem., 259(3):776-788, 1999.
Weyand and Goronzy, Ann. NY Acad. Sci., 987:140-149, 2003.
Wu and Wu, Biochemistry, 27:887-892, 1988.
Wu and Wu, J. Biol. Chem., 262:4429-4432, 1987.
Xanthou and Polihronis, Arthritis Rheum., 44(2):408-418, 2001.
Yang et al., Proc. Natl. Acad. Sci. USA, 87:9568-9572, 1990.
Zhang et al., J. Exp. Med., 203:141-152, 2006.

1. A method for inhibiting the binding of an antibody to an antigen in vivo comprising administering to a subject at least one peptide selected from the group consisting XL(X)ₘ, (X)ₘTP(X)ₙ (SEQ ID NO:1), (X)ₒY₁Y₂Y₃Y₄Y₅(X)ₚ (SEQ ID NO:2) and (X)ₒZ₁Z₂Z₃Z₄(X)ₚ (SEQ ID NO:3), or an analog thereof, wherein:
X is any amino acid;
Y₁-Y₅ are each any amino acid, so long as at least one of Y₁-Y₅ is a W residue, and the remain of Y₁-Y₅ comprises (i) Y, F or H residue, and (ii) D or E residue;
Z₁-Z₄ are each any amino acid, so long as at least one of Z₁-Z₄ is a W residue, and the remaining of Z₁-Z₄ comprises one D residue, two D residue's, one E residue, two E residue's, or one D and one E residue;
m and n are independently 0-3, but n+m must be two or greater; and
o and p are independently 0-2.

2. The method of claim 1, wherein said peptide has the formula (X)ₒZ₁Z₂Z₃Z₄(X)ₚ, and wherein said peptide comprises one D or E residue, and further comprises at one Y, F or H residue.

3. The method of claim 2, further comprising another second Y, F or H residue selected independent of said first Y, F or H residue.

4. The method of claim 1, wherein said peptide has the formula (x)ₒY₁Y₂Y₃Y₄Y₅(X)ₚ, and wherein said W residue is adjacent to said Y, F or H residue.

5. The method of claim 4, wherein said D or E residue is adjacent to said W residue or said Y, F or H residue.

6. The method of claim 1, wherein said subject suffers from an autoimmune disease.

7. The method of claim 6, wherein said autoimmune disease is SLE or rheumatoid arthritis.

8. The method of claim 1, further comprising administering to said subject at least two different peptides of the formula XLXXX (SEQ ID NO:4).

9. The method of claim 1, further comprising administering to said subject at least two different peptides of the formula (X)ₘTP(X)ₙ, or at least two different peptides of the formula (X)ₒZ₁Z₂Z₃Z₄(X)ₚ, or at least two different peptides of the formula (X)ₒY₁Y₂Y₃Y₄Y₅(X)ₚ, or

10-11. (canceled)

12. The method of claim 1, further comprising administering to said subject a peptide of the formula of the XLXXX and a peptide of the formula (X)ₒZ₁Z₂Z₃Z₄(X)ₚ or a peptide of the formula of the XLXXX and a peptide of the formula (X)ₒY₁Y₂Y₃Y₄Y₅(X)ₚ, or a peptide of the formula of the (X)ₒY₁Y₂Y₃Y₄Y₅(X)ₚ, and a peptide of the formula (X)ₒZ₁Z₂Z₃Z₄(X)ₚ.

13-14. (canceled)

15. The method of claim 1, further comprising administering to said subject a peptide of the formula of the (X)ₒY₁Y₂Y₃Y₄Y₅(X)ₚ and a peptide of the formula (X)ₘTP(X)ₙ.

16. The method of claim 1, further comprising administering to said subject a peptide of the formula of the XLXXX and a peptide of the formula (X)ₘTP(X)ₙ.

17. The method of claim 1, further comprising administering to said subject a peptide of the formula of the (X)ₒZ₁Z₂Z₃Z₄(X)ₚ and a peptide of the formula (X)ₘTP(X)ₙ.

18. The method of claim 1, further comprising administering to said subject a peptide of the formula of the (X)ₒZ₁Z₂Z₃Z₄(X)ₚ, a peptide of the formula (X)ₘTP(X)ₙ, a peptide of the formula (X)ₒY₁Y₂Y₃Y₄Y₅(X)ₚ, and a peptide of the formula XLXXX.

19. The method of claim 1, further comprising administering to said subject an anti-inflammatory or immunosuppressive agent other than said peptide.

20-24. (canceled)

25. An isolated and purified peptide selected from the group consisting XL(X)ₙ, (X)ₘTP(X)ₙ (SEQ ID NO:1), (X)ₒY₁Y₂Y₃Y₄Y₅(X)ₚ (SEQ ID NO:2) and (X)ₒZ₁Z₂Z₃Z₄(X)ₚ (SEQ ID NO:3), or an analog thereof, wherein:
X is any amino acid;
Z₁-Z₄ are each any amino acid, so long as at least one of Z₁-Z₄ is W, and the remaining of Z₁-Z₄ include one D, two D's, one E, two E's, or one D and one E;
m and n are independently 0-3, but n+m must be two or greater; and
o and p are independently 0-2.

26-27. (canceled)

28. An article of manufacture comprising:
(a) blood or plasma;
(b) a container for holding said blood or plasma; and
(c) at least one peptide selected from the group consisting XL(X)ₘ, (X)ₘTP(X)ₙ (SEQ ID NO:1), (X)ₒY₁Y₂Y₃Y₄Y₅(X)ₚ (SEQ ID NO:2) and (X)ₒZ₁Z₂Z₃Z₄(X)ₚ (SEQ ID NO:3), or an analog thereof bound to the inner surface of said container, wherein:
X is any amino acid;
Z₁-Z₄ are each any amino acid, so long as at least one of Z₁-Z₄ is W, and the remaining of Z₁-Z₄ include one D, two D's, one E, two E's, or one D and one E;
m and n are independently 0-3, but n+m must be two or greater; and
o and p are independently 0-2.

29. (canceled)

30. A method of detecting or removing a natural antibody from an antibody-containing fluid sample comprising contacting said sample with at least one peptide selected from the group consisting XL(X)ₘ, (X)ₘTP(X)ₙ (SEQ ID NO:1), (X)ₒY₁Y₂Y₃Y₄Y₅(X)ₚ (SEQ ID NO:2) and (X)ₒZ₁Z₂Z₃Z₄(X)ₚ (SEQ ID NO:3), or an analog thereof, wherein:
X is any amino acid;
Z₁-Z₄ are each any amino acid, so long as at least one of Z₁-Z₄ is W, and the remaining of Z₁-Z₄ include one D, two D's, one E, two E's, or one D and one E;
m and n are independently 0-3, but n+m must be two or greater; and
o and p are independently 0-2.

31. The method of claim 30, wherein said peptide is bound to a support.

32. (canceled)

33. The method of claim 30, wherein said peptide comprises a capture agent that permits binding of said capture agent to a support.

34-38. (canceled)
